(19) United States
(12) Patent Application Publication
(10) Pub. No.: US 2009/0259103 A1
(43) Pub. Date: Oct. 15, 2009

(54) Endoscope cooling device and endoscope system

(76) Inventor: Yasuo HIRATA, Tokyo (JP)

Correspondence Address: OSTROLENK FABER GERB & SOFFEN, 1180 AVENUE OF THE AMERICAS, NEW YORK, NY, 100368403, US

(21) Appl. No.: 12/101,421

(22) Filed: Apr. 11, 2008

(51) Int. Cl. A61B 1/12; A61B 1/01

(52) U.S. Cl. 600/114

(57) ABSTRACT

The endoscope cooling device for cooling an insertion portion having a bending portion that can be bent is provided with a guide tube into which the insertion portion including the bending portion is inserted to form a flow path of a cooling fluid between the insertion portion and the guide tube. The guide tube is provided with a cover portion for covering the bending portion, and other portions of the guide tube excluding the cover portion are more rigid than the cover portion.

### BACKGROUND OF THE INVENTION

1. Field of the Invention

The present invention relates to an endoscope cooling device for cooling an insertion portion of an endoscope and an endoscope system having the same.

2. Description of the Related Art

In order to observe a narrow part such as a duct for which an observer is otherwise unable to make a direct visual observation, conventionally there has been used an endoscope having an insertion portion that can be inserted into a test substance (for example, refer to the Japanese Published Unexamined Patent Application, First Publication No. 2005-342010). An observation portion such as a solid-state image sensor (CCD) is disposed at the proximal end of the insertion portion of the above-described endoscope, thus making it possible to observe the interior of the test substance. Further, an illuminating means used for lighting is provided at the distal end of the insertion portion, thus making it possible to favorably observe the test substance.

In this instance, since the insertion portion of the endoscope is provided as described above at the distal end with an observation portion such as a solid-state image sensor (for example, a CCD) and an illuminating devices, it is restricted to a maximum allowable working temperature of up to about 80° C. considering the heat-resistant temperatures of these devices. As a result, when the endoscope is used as an industrial endoscope in an attempt to observe the interior of an engine, the structure of which is complicated, it is impossible to make an observation as it is, with the insertion portion inserted therein, because the temperatures are raised to 200° C. or higher at the completion of an operation. Thus, the endoscope finds limited application. Therefore, an industrial endoscope has been proposed, which allows an operator to make an observation under the above high-temperature environment (for example, refer to Japanese Unexamined Patent Application, First Publication No. 2000-46482).

The industrial endoscope disclosed in the document of No. 2000-46482 is provided with an inner flexible body, an insertion portion having an outer flexible body provided by forming a space for the flowing of a fluid between itself and the inner flexible body, an outer casing fixed to the proximal end of the outer flexible body, the interior of which is communicatively connected to a space at which the fluid flows, and a valve fixed to the outer casing and allowing the fluid to flow into the outer casing. The valve is connected to a fluid supply device for supplying a cooling fluid by using a supply duct, thus allowing the cooling fluid to flow, by which the cooling fluid is released from the distal end from the interior of the outer casing through a space between the inner flexible body and the outer flexible body. Therefore, the endoscope can be used at high temperatures due to cooling by the cooling fluid.

### SUMMARY OF THE INVENTION

The endoscope cooling device of the present invention is an endoscope cooling device for cooling an insertion portion having a bending portion that can be bent, and provided with a guide tube into which the insertion portion including the bending portion is inserted to form a flow path of a cooling fluid between the insertion portion and the guide tube. The guide tube is provided with a cover portion for covering the bending portion, and other portions of the guide tube excluding the cover portion are more rigid than the cover portion.

In the endoscope cooling device of the present invention, the cover portion may be an elastic member.

In the endoscope cooling device of the present invention, a reinforcement member for reinforcing the guide tube may be provided at the above-described other portions.

In the endoscope cooling device of the present invention, a cooling lumen for allowing the cooling air to flow may be formed at the cover portion.

In the endoscope cooling device of the present invention, an operating lumen into which an operating fluid is supplied may be formed at the cover portion. The operating fluid is supplied to the operating lumen, by which the cover portion is bent.

In the endoscope cooling device of the present invention, the cover portion may be made thinner than the other portions.

In the endoscope cooling device of the present invention, a groove may be formed on an outer circumferential face of the cover portion.

In endoscope cooling device of the present invention, the cover portion is the endoscope cooling device described in claim 1, which is made of a tube wound in a helical shape, thus allowing the cooling fluid to flow through the tube.

In the endoscope cooling device of the present invention, the cover portion is made of a tube wound in a coil shape, thus allowing the cooling fluid to flow through the tube.

In the endoscope cooling device of the present invention, the cover portion is the endoscope cooling device described in claim 1, which is formed in a bellows shape.

The endoscope cooling device of the present invention may be provided with a fluid supply means for supplying the cooling fluid to the guide tube.

The endoscope cooling device of the present invention may be provided with a regulating portion for regulating movement of the distal end portion of the insertion portion with respect to the guide tube.

In the endoscope cooling device of the present invention, the regulating portion may regulate rotational movement of the distal end portion around the central axis with respect to the guide tube.

In the endoscope cooling device of the present invention, the regulating portion may regulate back and forth movement of the guide tube in the lengthwise direction thereof.

In the endoscope cooling device of the present invention, the regulating portion may be provided with a raised portion provided at one end of either the guide tube or the distal end portion and a recessed portion at the other end of either the guide tube or the distal end portion with which the raised portion is engaged.

In the endoscope cooling device of the present invention, the regulating portion may be provided with an auxiliary member fitted into the distal end portion. In this instance, the raised portion or the recessed portion is preferably formed on the auxiliary member.

In the endoscope cooling device of the present invention, the guide tube may be provided with an inner sheath into which the insertion portion including the bending portion is inserted to form a first flow path of a cooling fluid between the outer circumferential face of the insertion portion and the inner circumferential face of the guide tube, and an outer sheath into which the inner sheath is inserted to form a second flow path of the cooling fluid between the outer circumferential face of the inner sheath and the inner circumferential face of the guide tube. At this time, it is preferable that the cover portion is provided respectively on the inner sheath and the outer sheath.

In the endoscope cooling device of the present invention, the guide tube can be divided into a plurality of portions, and one of the divided portions or some of the divided portions may be used as the guide tube.

The endoscope system of the present invention is provided with the above-described endoscope cooling device and an endoscope having the insertion portion.

### BRIEF DESCRIPTION OF THE DRAWINGS

FIG. 1 is a view showing a first embodiment of the endoscope cooling device according to the present invention, or an overall block diagram showing a constitution of a heat-resistant endoscope, which is used together with an endoscope.

FIG. 2 is an enlarged perspective view showing the guide tube shown in FIG. 1.

FIG. 3 is an enlarged view of the guide tube shown in FIG. 1, or a side sectional view showing the distal end side further than a fixing ring.

FIG. 4 is an enlarged side-sectional view showing the back end portion of the guide tube shown in FIG. 1.

FIG. 5 is a side sectional view showing a state in which the fixing ring shown in FIG. 4 is coupled to the rear base portion.

FIG. 6 is a side sectional view showing a state in which an elastic ring is reduced in diameter by moving the fixing ring shown in FIG. 5 to the distal end of the guide tube.

FIG. 7 is a view showing an exemplified variation of the insertion portion of the endoscope shown in FIG. 1, or a perspective view showing a state in which a male threaded portion is provided in the vicinity of the back end of the bending portion.

FIG. 8 is a view showing an exemplified variation of the guide tube shown in FIG. 1, or a perspective view showing a state in which a female threaded portion is provided at the distal end of the rear base portion.

FIG. 9 is a view showing an exemplified variation of the insertion portion of the endoscope and the guide tube shown in FIG. 1, or a view showing a state in which a hole portion is formed at the insertion portion of the endoscope and a through hole is formed on the guide tube.

FIG. 10 is a view showing a state in which the guide tube shown in FIG. 9 is fixed to the insertion portion of the endoscope.

FIG. 11 is a view showing major portions of a second embodiment of the endoscope cooling device of the present invention.

FIG. 12 is a view showing major portions of a third embodiment of the endoscope cooling device of the present invention.

FIG. 13 is a perspective view showing major portions of a fourth embodiment of the endoscope cooling device of the present invention.

FIG. 14 is a view showing an exemplified variation of the cover portion shown in FIG. 13, or a perspective view showing a state in which a cooling air supply tube is formed in a coil shape.

FIG. 15 is a view showing an exemplified variation of the cover portion shown in FIG. 13, or a perspective view showing a state in which a groove is formed at the cover portion.

FIG. 16 is a view showing an exemplified variation of the cover portion shown in FIG. 13, or a perspective view showing a state in which a bellows portion is formed at the cover portion.

FIG. 17 is a view showing major portions of fifth embodiment of the endoscope cooling device of the present invention, or a perspective view showing a state in which the cover portion is broken.

FIG. 18 is a view showing sixth embodiment of the endoscope system of the present invention, or an exploded perspective view of the distal end portion of a sheath which constitutes the endoscope cooling device included in the present embodiment.

FIG. 19 is a side sectional view showing the sheath of the endoscope cooling device included in the sixth embodiment.

FIG. 20 is a side sectional view showing the sheath of the endoscope cooling device included in the sixth embodiment, which is bent.

FIG. 21 is a view showing a first exemplified variation of the sixth embodiment, or an exploded perspective view of the distal end portion of the sheath which constitutes the endoscope cooling device of the exemplified variation.

FIG. 22 is a side sectional view showing the sheath of the endoscope cooling device included in the first exemplified variation.

FIG. 23 is a view showing a second exemplified variation of the sixth embodiment, or an exploded perspective view of the distal end portion of the sheath which constitutes the endoscope cooling device included in the exemplified variation.

FIG. 24 is a view showing a seventh embodiment of the endoscope system of the present invention, or an exploded perspective view of the distal end portion of the sheath which constitutes the endoscope cooling device included in the present embodiment.

FIG. 25 is a view showing an eighth embodiment of the endoscope system of the present invention, or an exploded perspective view of the distal end portion of the sheath which constitutes the endoscope cooling device included in the present embodiment.

FIG. 26 is a side sectional view showing the sheath of the endoscope cooling device included in the eighth embodiment.

FIG. 27 is a view showing a ninth embodiment of the endoscope system of the present invention, or an exploded perspective view of the distal end portion of the sheath which constitutes the endoscope cooling device included in the present embodiment.

FIG. 28 is an overall block diagram showing a tenth embodiment of the endoscope system of the present invention.

FIG. 29 is a side sectional view showing the sheath of the endoscope cooling device included in the tenth embodiment.

FIG. 30 is an exploded perspective view showing the sheath of the endoscope cooling device included in the tenth embodiment.

FIG. 31 is a view showing an eleventh embodiment of the endoscope system of the present invention, or an exploded perspective view showing the distal end portion of the sheath which constitutes the endoscope cooling device included in the present embodiment.

FIG. 32 is an exploded perspective view showing the sheath of the endoscope cooling device included in the eleventh embodiment.

FIG. 33 is a side sectional view showing the sheath of the endoscope cooling device included in the eleventh embodiment.

FIG. 34 is a view showing a twelfth embodiment of the endoscope system of the present invention, or a side sectional view showing the sheath which constitutes the endoscope cooling device included in the present embodiment.

FIG. 35 is an exploded perspective view showing the sheath of the endoscope cooling device included in the twelfth embodiment.

FIG. 36 is a block diagram showing major portions of the endoscope cooling device related to a thirteenth embodiment of the present invention.

FIG. 37 is a block diagram showing major portions of the endoscope cooling device related to a fourteenth embodiment of the present invention.

FIG. 38 is a block diagram showing major portions of the endoscope cooling device related to a fifteenth embodiment of the present invention.

FIG. 39A is a perspective view showing major portions of the endoscope cooling device related to a sixteenth embodiment of the present invention, which includes a partial section.

FIG. 39B is a perspective view showing major portions of the endoscope cooling device related to the sixteenth embodiment of the present invention, which is viewed in a direction different from FIG. 39A.

FIG. 40 is a sectional view showing major portions of the endoscope cooling device related to the sixteenth embodiment of the present invention.

### DESCRIPTION OF THE PREFERRED EMBODIMENTS

### First Embodiment

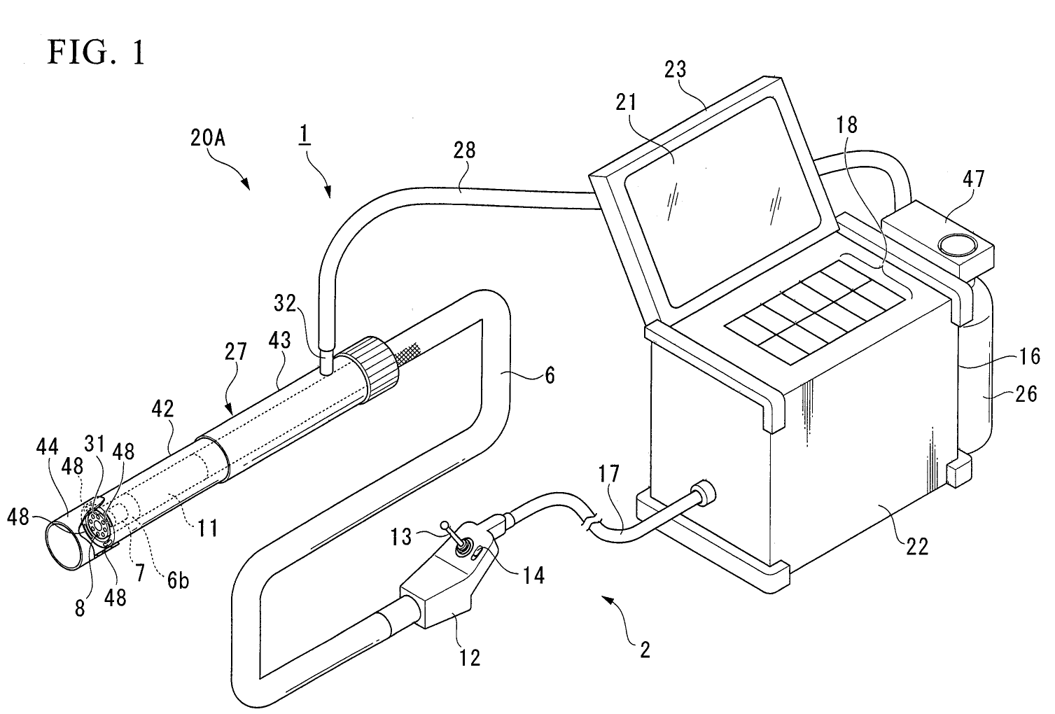

An explanation will be made for a first embodiment of the endoscope system of the present invention with reference to FIG. 1 to FIG. 6.

As shown in FIG. 1, the endoscope system of the present embodiment is provided with a direct-viewing-type endoscope 1 and an endoscope cooling device 20A, which allows a cooling fluid such as air and water to flow in an insertion portion 6 of the endoscope 1, thereby cooling the distal end of the insertion portion 6.

The endoscope 1 is provided with an insertion portion 6 inserted into a test substance, an operating portion 12 for operating the insertion portion 6 in various ways and a device main body 16 connected to the operating portion 12 via a universal cord 17. The insertion portion 6 has a long body, the distal end of the body is provided with an observation portion 7 having a built-in CCD or others and a distal end portion 5 having an illumination portion 8 for irradiating light for illumination to the test substance. A bending portion 11 that can be bent is provided in the vicinity of the distal end portion 5. The bending portion 11 is bent, by which the distal end of the insertion portion 6 can be pointed at any desired direction.

A joy stick 13 for operating the insertion portion 6 in a bending manner is provided at the operating portion 12. Further, an air supply switch 14 is provided at the operating portion 12. The air supply switch 14 is depressed, by which cooling air is supplied from a compressor 26 to be described later. The device main body 16 is provided with a box portion 22 and a lid portion 23 attached to the box portion 22 so as to be opened and closed. An operating button 18 for conducting various types of settings and operations is provided at the box portion 22. A display portion 21 made of a liquid crystal panel or the like is provided at the lid portion 23. An image taken by the observation portion 7 is displayed on the display portion 21.

The endoscope cooling device 20A is provided with a compressor 26 for compressing and feeding cooling air (a cooling fluid) and a tubular guide tube 27. The compressor 26 and the guide tube 27 are connected to each other via a flexible cooling air supply tube (flowing tube). A valve 47 is provided at the compressor 26, and the valve 47 is opened, by which the cooling air is fed from the compressor 26 to the cooling air supply tube 28.

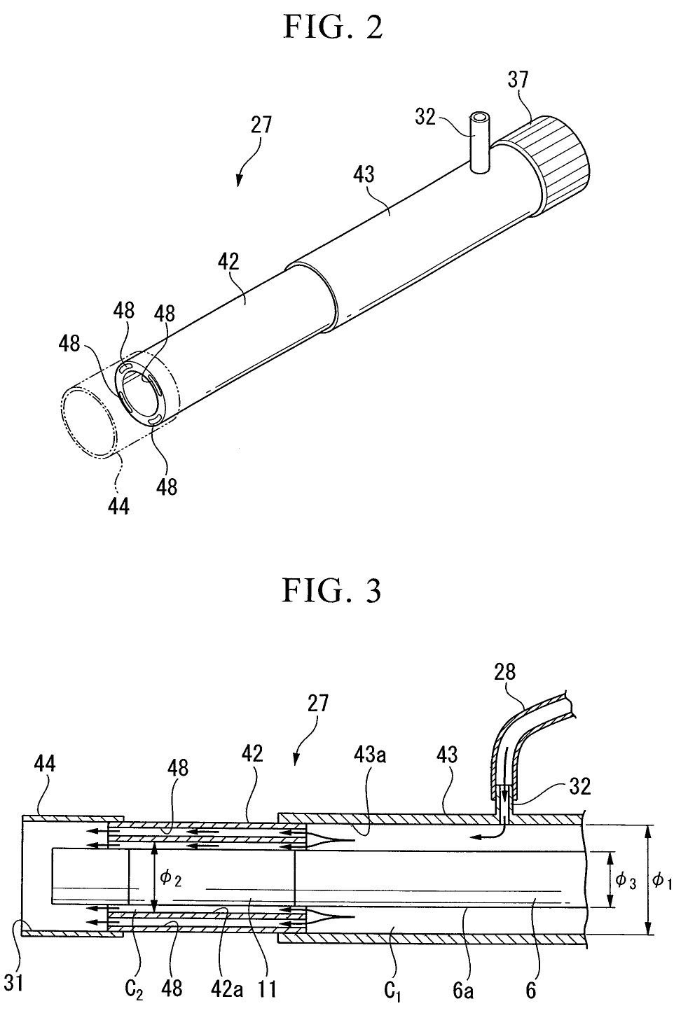

A cylindrical hole (insertion hole) 31 is formed on the guide tube 27, and the insertion portion 6 is inserted into the cylindrical hole 31. Since the guide tube 27 is shorter than the insertion portion 6, the guide tube 27 having the insertion portion 6 inserted into the cylindrical hole 31 is allowed to move along the length direction of the insertion portion 6. As shown in FIG. 2, the guide tube 27 is provided with a cylindrical cover portion 42 for covering the bending portion 11 when the guide tube 27 is fixed to the insertion portion 6, a cylindrical proximal end base 43 coupled to the back end of the cover portion 42 and a cylindrical distal end base 44 provided at the distal end of the cover portion 42. The back end of the cover portion 42 is fixed to an opening portion at the distal end of the proximal end base 43 by bonding, for example, and bonded in such a manner that the cover portion 42 and the proximal end base 43 are arranged on the same axial line. The distal end of the cover portion 42 is fixed to an opening portion at the back end of the distal end base 44 by bonding, for example, and bonded in such a manner that the cover portion 42 and the proximal end base 44 are arranged on the same axial line.

The cover portion 42 is made of an elastic material having heat resistance and flexibility such as silicon and easily bent by application of an external force. On the other hand, the proximal end base 43 is made of a material such as metal much higher in rigidity than the cover portion 42. In other words, the cover portion 42 is made more flexible than the proximal end base 43. Four cooling lumens 48 extending in the lengthwise direction of the cover portion 42 are formed at a wall portion of the cover portion 42. These four cooling lumens 48 are arranged so as to be spaced at equal intervals in the circumferential direction of the cover portion 42. Both ends of the cooling lumen 48 are opened.

As shown in FIG. 3, the inner diameter Ø₁ of the proximal end base 43 is set to be larger than the inner diameter Ø₂ of the cover portion 42, and the inner diameter Ø₂ of the cover portion 42 is set to be larger than the outer diameter Ø₃ of the insertion portion 6. Therefore, when the insertion portion 6 is inserted into the cylindrical hole 31, a clearance C₁ is formed between an outer circumferential face 6a of the insertion portion 6 and an inner circumferential face 43a of the proximal end base 43, and a clearance C₂ is also formed between the outer circumferential face 6a of the insertion portion 6 and an inner circumferential face 42a of the cover portion 42.

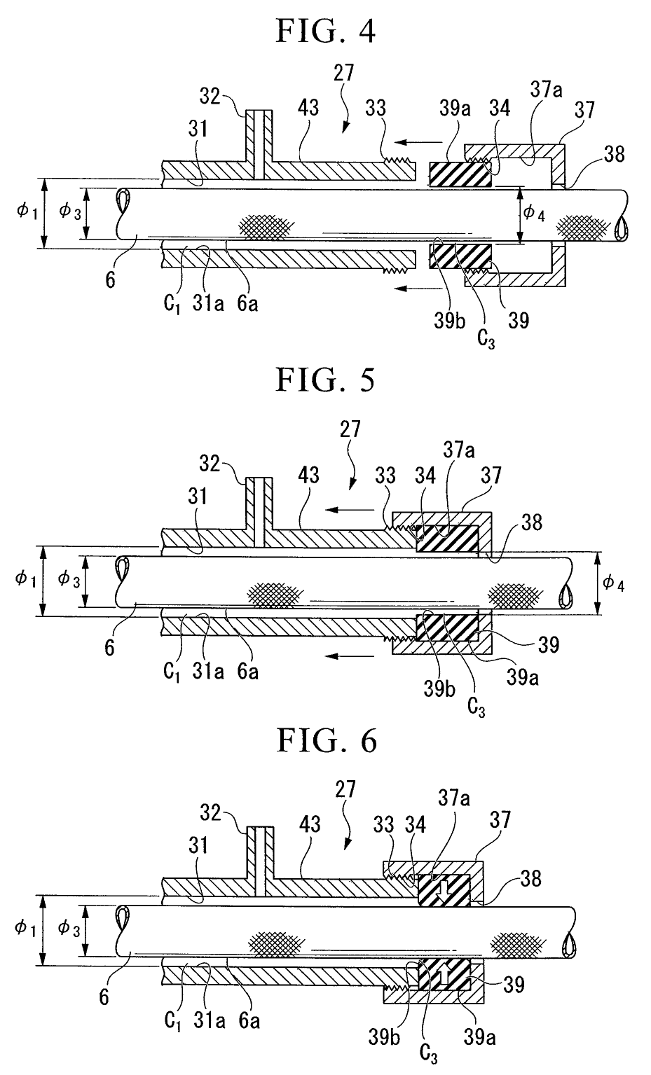

A cooling fluid supply port (a coupling portion for cooling) 32 leading to the cylindrical hole 31 is provided at the back end portion of the proximal end base 43. The above-described cooling air supply tube 28 is attached to the cooling fluid supply port 32. Further, as shown in FIG. 4, a closed-end cylindrical fixing ring 37 is coupled in a removable manner to the back end of the proximal end base 43. In other words, a male threaded portion 33 is formed on the outer circumferential face at the back end portion of the proximal end base 43, while a female threaded portion 34 is formed on the inner circumferential face at the open end portion of the fixing ring 37, and the male threaded portion 33 is screwed with the female threaded portion 34. Next, upon rotation of the fixing ring 37, the threading position at which the male threaded portion 33 is screwed with the female threaded portion 34 is shifted, by which the fixing ring 37 moves to the length direction of the proximal end base 43, that is, in a direction at which the back end face of the proximal end base 43 is brought closer to or spaced away from the bottom face of the fixing ring 37. A hole 38 is formed on the bottom face of the fixing ring 37, and the insertion portion 6 is inserted into the hole 38.

Further, an annular elastic ring 39 made of an elastic member is provided inside the fixing ring 37. The outer diameter of the elastic ring 39 is set to be equal to or larger than the inner diameter of the fixing ring 37, while the inner diameter Ø₄ of the elastic ring 39 is set to be larger than the outer diameter Ø₃ of the insertion portion 6. Therefore, the elastic ring 39 is arranged inside the fixing ring 37 in a state that the fixing ring 37 is removed from the proximal end base 43 and the fixing ring 37 is fixed to the proximal end base 43, by which the outer circumferential face 39a of the elastic ring 39 is in contact with the inner circumferential face 37a of the fixing ring 37. Further, when the insertion portion 6 is inserted into the fixing ring 37 via the elastic ring 39 in a state in which the outer circumferential face 39a of the elastic ring 39 is in contact with the inner circumferential face 37a of the fixing ring 37, a clearance C₃ is formed between the outer circumferential face 6a of the insertion portion 6 and the inner circumferential face 39b of the elastic ring 39.

Further, the fixing ring 37 is coupled to the back end portion of the proximal end base 43 and allowed to move to a predetermined position of the proximal end base 43 in a state in which the outer circumferential face 39a of the elastic ring 39 is in contact with the inner circumferential face 37a of the fixing ring 37. Next, as shown in FIG. 5, the front end face of the elastic ring 39 is in contact with the back end face of the proximal end base 43, and the back end face of the elastic ring 39 is in contact with the bottom face of the fixing ring 37.

Next, a description is given for actions of the thus constituted endoscope cooling device 20A according to the present embodiment.

First, the guide tube 27 is attached so as to cover the distal end portion 5 of the insertion portion 6. After the insertion portion 6 is inserted from the back end of the proximal end base 43 into the hole 38 of the fixing ring 37 and the elastic ring 39, the distal end portion 5 is arranged inside the cylindrical hole 31 of the guide tube 27 in such a manner that the cover portion 42 covers the bending portion 11. Next, upon rotation of the fixing ring 37, the back end face of the proximal end base 43 is brought closer to the bottom face of the fixing ring 37. As shown in FIG. 6, the elastic ring 39 is pressed back and forth by the bottom face of the fixing ring 37 and the back end face of the proximal end base 43. At this time, the outer circumferential face 39a of the elastic ring 39 is in contact with the inner circumferential face 37a of the fixing ring 37, and the elastic ring 39 is regulated in undergoing elastic deformation so as to expand the diameter. However, since a clearance C₃ is formed between the inner circumferential face 39b of the elastic ring 39 and the outer circumferential face 6a of the insertion portion 6, the elastic ring 39 elastically deforms inwardly so as to reduce the inner diameter. As a result, the outer circumferential face 6a of the insertion portion 6 is firmly attached to the inner circumferential face 39b of the elastic ring 39 all across the circumference, thereby regulating the movement of the guide tube 27 and also sealing the guide tube 27 and the insertion portion 6 in an air-tight manner. Thereby, the guide tube 27 is fixed to the insertion portion 6.

After the guide tube 27 is fixed to the insertion portion 6 as described above, the insertion portion 6 is inserted into a test substance, together with the guide tube 27. Next, the illumination portion 8 is used to illuminate the interior of the test substance, and obtain an image illuminated by the illumination light by the observation portion 7. Further, the image obtained by the observation portion 7 is displayed on the display portion 21. While viewing the image displayed on the display portion 21, an operator manipulates the joy stick 13 to change the direction of the distal end of the insertion portion 6, thereby examining the interior of the test substance.

Further, where the test substance is held at high temperatures, like an engine immediately after use, the temperature may exceed the maximum allowable temperature at the observation portion 7 or the illumination portion 8, thus the normal operation of the observation portion 7 and the illumination portion 8 may be failed. Therefore, an air supply switch 14 is depressed to open a valve 47, thereby supplying cooling air to the guide tube 27 from the compressor 26. In other words, cooling air inside the compressor 26 is supplied into the cylindrical hole 31 of the guide tube 27 via the cooling air supply tube 28 and the cooling fluid supply port 32. Since the back end of the proximal end base 43 is sealed by the elastic ring 39 in an air-tight manner, the cooling air supplied inside the cylindrical hole 31 is, as shown in FIG. 3, fed to the distal end of the guide tube 27 through the clearance C₁ formed between the insertion portion 6 and the proximal end base 43. Thereafter, the cooling air reaches the cover portion 42, and is ejected from the distal end of the guide tube 27 through a cooling lumen 48 and a clearance C₂. The cooling air cools the distal end portion 5 of the insertion portion 6 to protect the observation portion 7 and the illumination portion 8.

In this case, since air passes through the clearance C₂ between the insertion portion 6 and the guide tube 27 and through the cooling lumen 48 outside thereof, the observation portion 7 and the illumination portion 8 are reliably protected from external high-temperature environments. In other words, air passing through the lumen 48 provides a heat-insulating barrier against external environments, thereby reducing heat transmitted inside. Air also flows through the inside of the clearance C₂, to form a heat-insulating barrier, which is double-layered, thereby reducing the heat transmitted inside.

Conventionally, when the fixed insertion portion 6 at which the guide tube 27 is fixed is inserted into a test substance, the guide tube 27 contracts in the lengthwise direction or the distal end deviates from an observation position. However, in the present embodiment, since the proximal end base 43 and the distal end base 44 are made of a metal, there is no chance that the proximal end base 43 or the distal end base 44 contracts or that the distal end deviates from an observation position upon insertion of the insertion portion 6. Therefore, the insertion portion 6 can be easily inserted into the test substance. Further, the bending portion 11 is covered by a flexible cover portion 42 made of an elastic material when the bending portion 11 is bent. Therefore, the cover portion 42 is also bent accordingly when the bending portion 11 is bent.

According to the endoscope cooling device 20A of the present embodiment, since the proximal end base 43 and the distal end base 44 are made of a metal, the front and back portions of the guide tube 27 is secured for the rigidity. Since the bending portion 11 is covered with a flexible cover portion 42 made of an elastic material, the bending portion 11 can be easily bent. In other words, a test substance can be easily inserted, while the insertion portion 6 is kept so as to be bendable.

Further, when the insertion portion 6, the outer diameter of which is equal to the inner diameter Ø₂ of the cover portion 42, is inserted into the cylindrical hole 31, cooling air is less likely to flow due to the absence of the clearance C₂. However, in the present embodiment, cooling air can be supplied to the distal end portion 5 of the insertion portion 6 only by the cooling lumen 48 of the cover portion 42. Therefore, the present embodiment is able to cope with various types of insertion portions 6 different in the outer diameters thereof without using a plurality of guide tubes 27 different in dimension. Further, since a fluid layer is formed by the cooling lumen 48 on an outer circumference of the insertion portion 6, it is possible to protect the insertion portion 6 from high temperatures.

In addition, in the present embodiment, four cooling lumens 48 are provided at the cover portion 42. There is, however, no restriction on the number of cooling lumens 48, which may be changed, if necessary. In addition, no cooling lumen 48 may be provided under the conditions that a clearance is secured between the cover portion 42 and the insertion portion 6. However, it is preferable to provide the cooling lumen 48 because a large quantity of cooling air can flow through the cooling lumens.

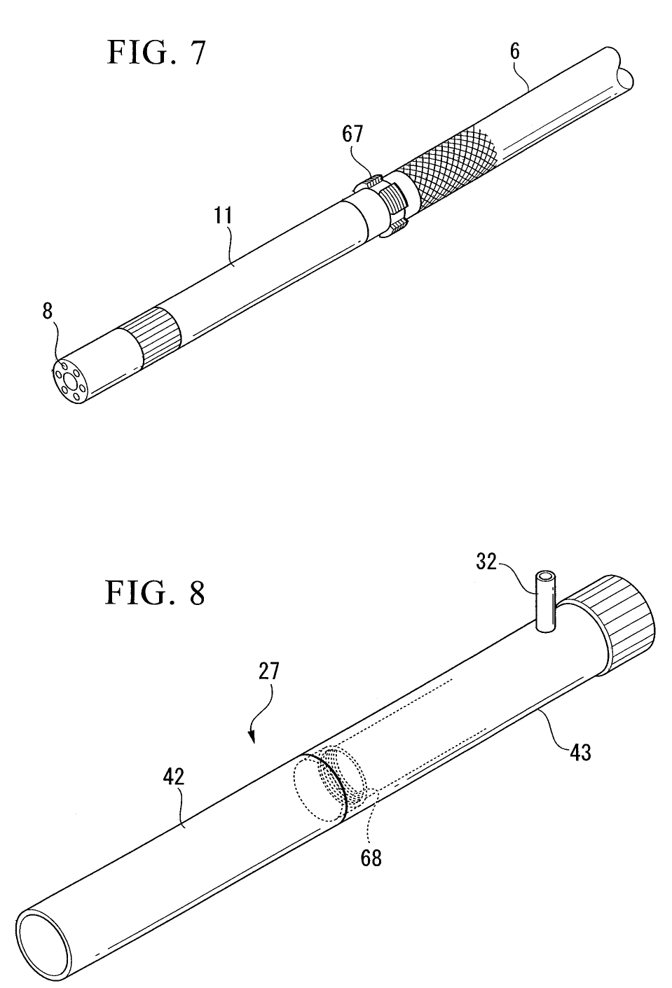

Still further, in the present embodiment, the guide tube 27 is fixed to the fixing ring 37. However, there is no restriction on a structure for fixing the guide tube 27, which may appropriately be changed. For example, as shown in FIG. 7 and FIG. 8, a male threaded portion 67 may be provided in the vicinity of the back end of the bending portion 11 at the insertion portion 6, and a female threaded portion 68 may be made at the distal end of the proximal end base 43. Next, the male threaded portion 67 is screwed into the female threaded portion 68, by which the guide tube 27 is fixed to the insertion portion 6. The male threaded portion 67 is divided into a plurality of portions, and cooling air passes through a clearance formed between the thus divided male threaded portions 67, heading toward the distal end of the insertion portion 6. Thereby, the guide tube 27 is securely prevented from positional deviation. Further, the guide tube 27 is fixed in the vicinity of the back end of the bending portion 11 to smoothly prevent a deviation of the guide tube 27, which occurs depending on the bending of the bending portion 11. Thereby, the bending portion 11 can be bent more easily.

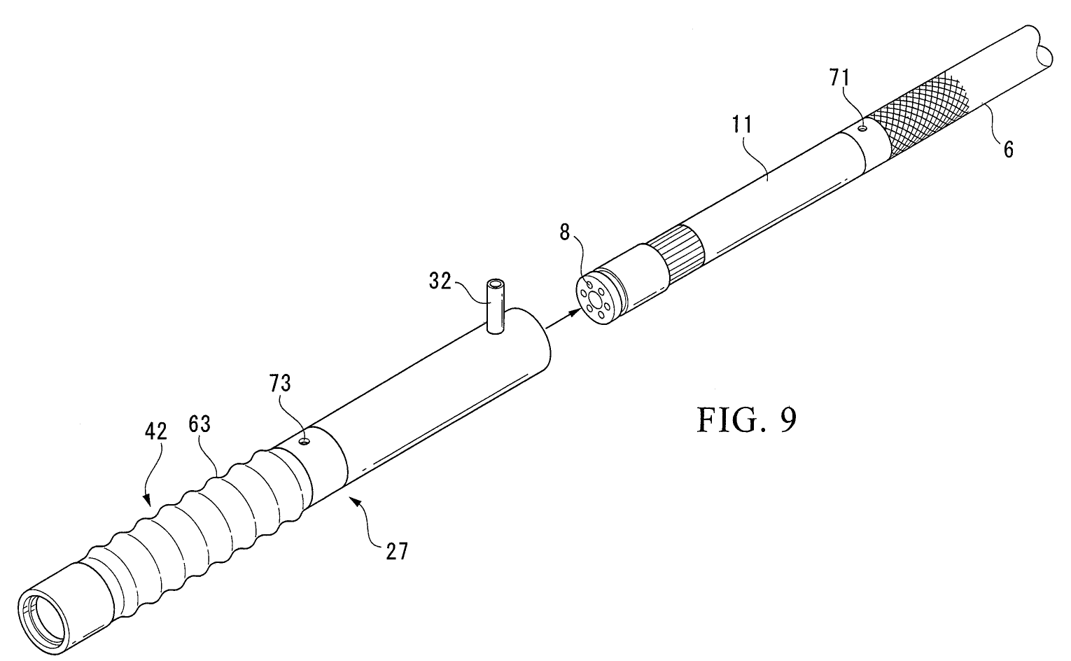

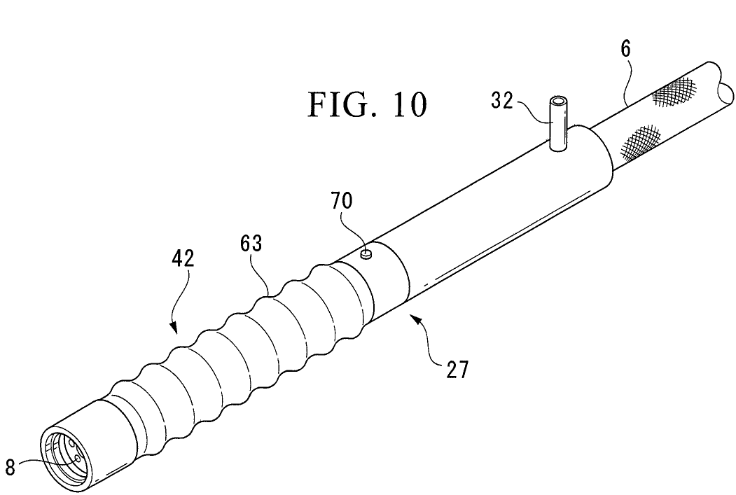

Further, as shown in FIG. 9 and FIG. 10, a hole portion 71 may be formed in the vicinity of the back end at the bending portion 11 of the insertion portion 6, and a through hole 73 may be provided in the vicinity of the back end at the cover portion 42 of the guide tube 27. Next, the guide tube 27 is arranged at the distal end of the insertion portion 6, the hole portion 71 is allowed to align with the through hole 73, and a pin 70 is inserted into the hole portion 71 via the through hole 73. Thereby, the guide tube 27 is fixed to the insertion portion 6, and the guide tube 27 is securely prevented from positional deviation.

Still further, in the present embodiment, a fixing ring 37 is used to fix the guide tube 27 to the insertion portion 6. However, the guide tube 27 may be fixed to the operating portion 12 so as to be extended from the operating portion 12 to the distal end portion 5 of the insertion portion 6. It is preferable to provide the fixing ring 37 because the ring responds to various types of insertion portions 6 which are different in length.

### Second Embodiment

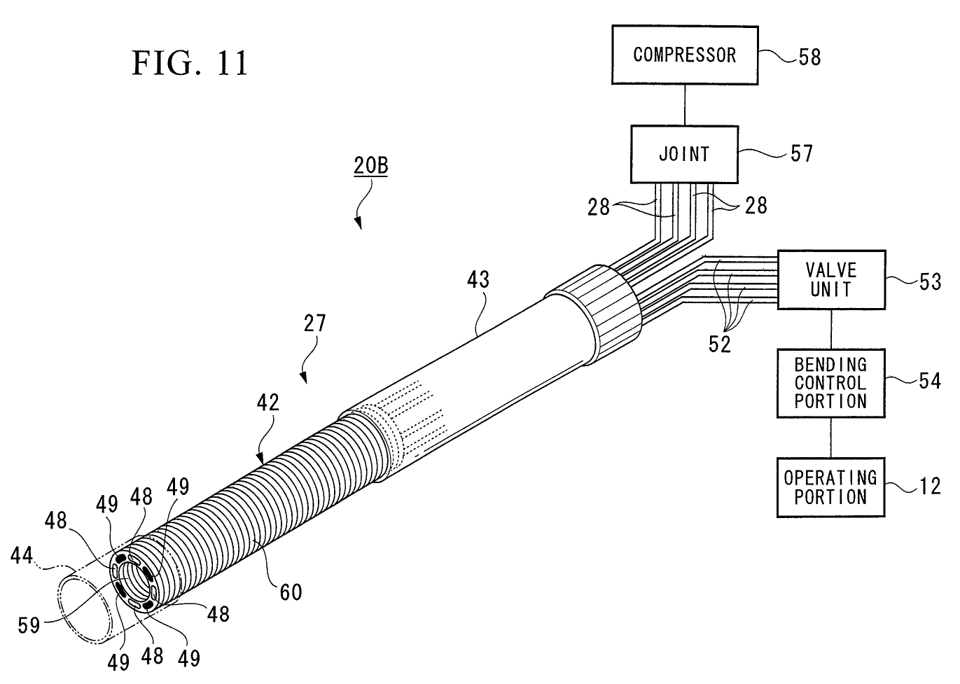

Next, a description is given for a second embodiment of the endoscope system of the present invention with reference to FIG. 11. In the present embodiment, members common to those used in the above-described embodiment will be given the same reference numerals, the explanation of which will be omitted here.

In the endoscope cooling device 20B of the present embodiment, an operating lumen 49 into which operating air is supplied is formed at the circumferential wall portion of the cover portion 42. The operating lumens 49 and cooling lumens 48 are formed respectively in four units, and the operating lumens 49 and the cooling lumens 48 are alternately arranged in the circumferential direction of the guide tube 27. The distal end of each of the operating lumens 49 is sealed by an adhesive agent or the like in an air-tight manner, and an operating air supply tube 52 is connected to the back end of each of the operating lumens 49 in an air-tight manner. Further, an inner coil 59 is provided inside the cover portion 42 along the inner circumferential face, and an outer coil 60 is provided outside the cover portion 42 along the outer circumferential face.

A valve unit 53 for selectively supplying operating air to the four operating lumens 49 is coupled to the back end of the operating air supply tube 52. A bending control portion 54 for controlling the opening and closing of a valve of the valve unit 53 depending on the operation of the operating portion 12 is electrically connected to the valve unit 53. Further, the valve unit 53 is connected to a compressor 58 for supplying air.

Further, one end of the cooling air supply tube 28 is connected to the back end of the cooling lumen 48 in an air-tight manner. The other end of the cooling air supply tube 28 is connected to a joint 57 for discharging cooling air after divergence. In the present embodiment, the cooling air supply tube 28 functions as a cooling coupling portion. The joint 57 is connected to the compressor 58. The compressor 58 is driven to supply air to the joint 57 and the valve unit 53. Air supplied to the joint 57 is supplied to the cooling lumens 48 via the cooling air supply tube 28. The air supplied to the cooling lumens 48 is ejected from the distal end of the guide tube 27. Cooling air flows through the cooling lumens 48, thereby cooling the distal end portion 5 of the insertion portion 6, which is inserted into the guide tube 27, thereby protecting the observation portion 7 and the illumination portion 8.

In the above-constituted endoscope cooling device 20B, when the operating portion 12 is operated, any one of the valves of the valve unit 53 is opened by the bending control portion 54, by which operating air is supplied to any one of the operating lumens 49 from the compressor 58 via the operating air supply tube 52. The inner coil 59 and the outer coil 60 respectively support the interior of the cover portion 42 and the exterior of the cover portion 42. Therefore, when the operating air is supplied to any one of the operating lumens 49, the operating lumen 49 concerned is extended in the lengthwise direction, and the cover portion 42 is bent. Thereby, the distal end of the guide tube 27 is changed in direction. In other words, the operating air is selectively supplied to any one of the four operating lumens 49, thus making it possible to point the distal end of the guide tube 27 to a desired direction.

According to the endoscope cooling device 20B of the present embodiment, the guide tube 27 itself can be bent, without depending on the bending operation of the bending portion 11, thus making it possible to favorably retain the bending performance of the bending portion 11 of the insertion portion 6.

### Third Embodiment

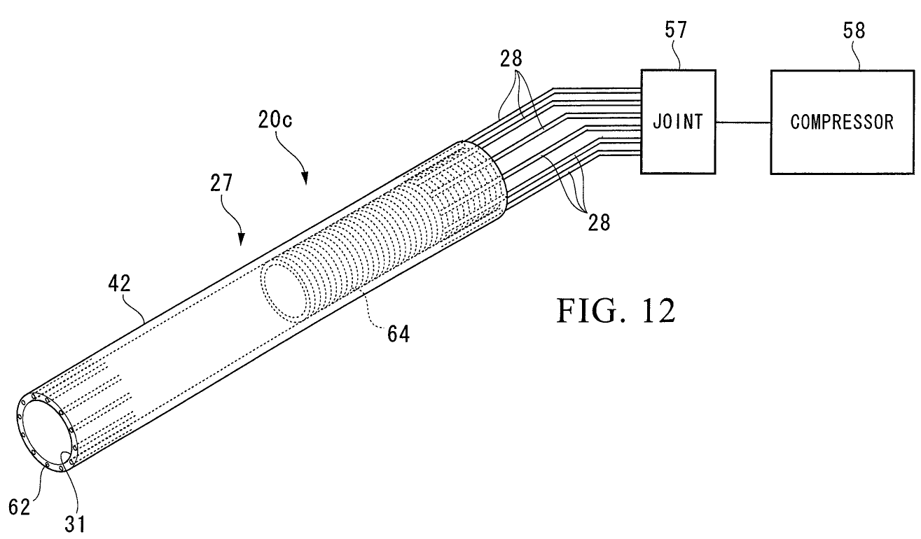

Next, a description is given for a third embodiment of the endoscope system of the present invention with reference to FIG. 12. In the present embodiment, members common to those used in the above-described embodiment will be given the same reference numerals, the explanation of which will be omitted here.

In the endoscope cooling device 20C of the present embodiment, the guide tube 27 is made of an elastic material such as silicon and composed of a single component extended in a cylindrical shape. A firmly-attached coil for reinforcement (reinforcement member) 64, which is extended from the back end to the central part in the lengthwise direction, is provided inside the guide tube 27. Thereby, an increased rigidity is imparted to the rear half of the guide tube 27, while the front half of the guide tube 27 is made flexible in comparison with the rear half.

A plurality of ducts 62 extended in the lengthwise direction are formed at the circumferential wall portion of the guide tube 27. The cooling air supply tube 28 is connected to the back end of the duct 62 in an air-tight manner.

With the endoscope cooling device 20C of the present embodiment, flexibility can be imparted to a cover portion of the guide tube 27, and also rigidity of the guide tube 27 (excluding the cover portion) can be secured, although it is simple in structure.

In the present embodiment, the firmly-attached coil 64 is extended from the back end of the guide tube 27 to the central part in the lengthwise direction. However, there is no restriction on the place at which the firmly-attached coil 64 is provided. The place may appropriately be changed, depending on the length of the guide tube 27 or the shape of a test substance. For example, the firmly- attached coil 64 may be extended from the back end of the guide tube 27 to the front at the central part or extended from the back end to a place over the central part.

### Fourth Embodiment

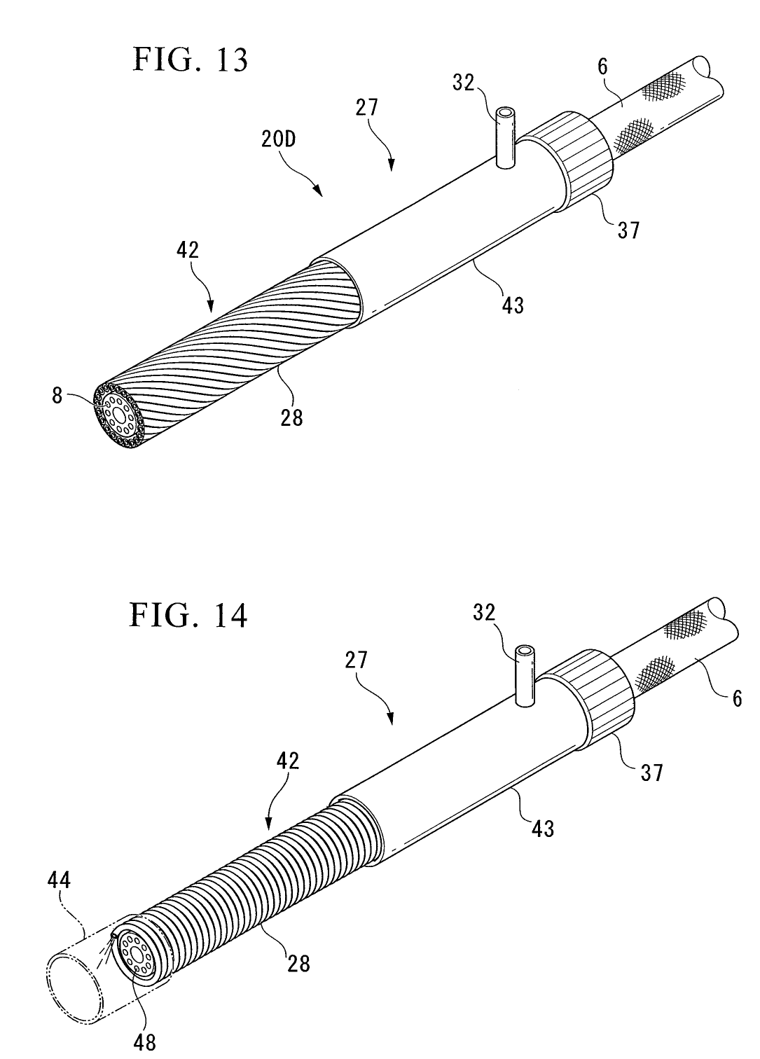

Next, a description is given for a fourth embodiment of the endoscope system of the present invention with reference to FIG. 13. In the present embodiment, members common to those used in the above-described embodiment will be given the same reference numerals, the explanation of which will be omitted here.

In the endoscope cooling device 20D of the present embodiment, the cover portion 42 is made of a plurality of cooling air supply tubes 28 wound in a helical shape. The distal end face of each of the cooling air supply tubes 28 is aligned in parallel with a face perpendicular to the length direction of the guide tube 27, and cooling air is discharged forward from the distal end of the cooling air supply tube 28. According to the endoscope cooling device 20D of the present embodiment, the cover portion 42 can be bent easily.

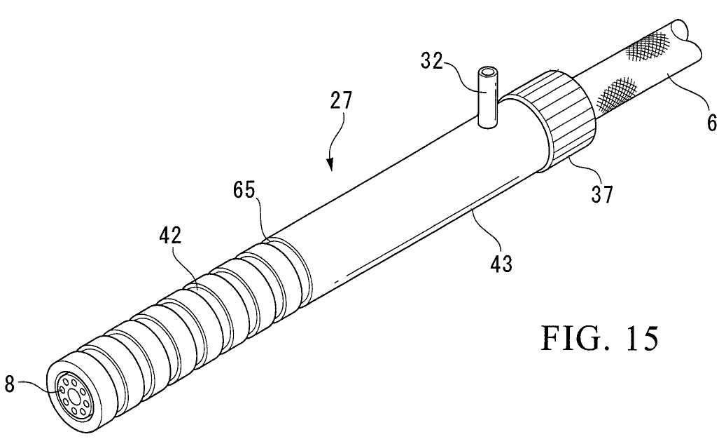

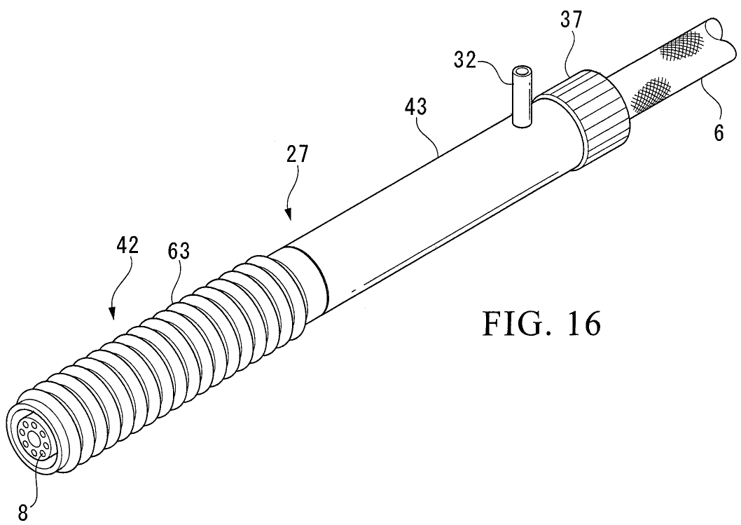

In the present embodiment, the cover portion 42 is composed of a plurality of the cooling air supply tubes 28 wound in a helical shape. However, there is no particular restriction on the structure of the cover portion 42, and the structure can appropriately be changed. For example, as shown in FIG. 14, the cover portion 42 may be composed of one or several cooling air supply tubes 28 wound in a coil shape. Further, as shown in FIG. 15, a plurality of grooves 65 formed circumferentially or a helical groove may be provided on the outer circumferential face of the cover portion 42. Still further, as shown in FIG. 16, a bellows portion 63 may be provided at the cover portion 42. Thereby, the cover portion 42 can be bent easily.

### Fifth Embodiment

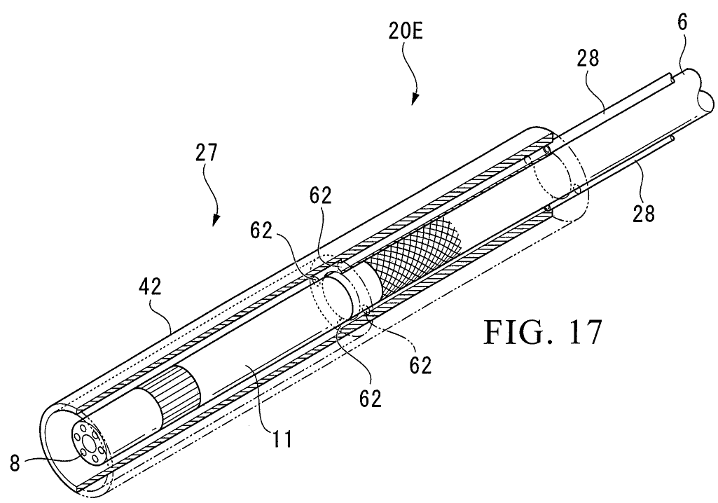

Next, a description is given for a fifth embodiment of the endoscope system of the present invention with reference to FIG. 17. In the present embodiment, members common to those used in the above-described embodiment will be given the same reference numerals, the explanation of which will be omitted here.

In the endoscope cooling device 20E of the present embodiment, the guide tube 27 is made of a single component extended in a cylindrical shape. The outer diameter of the guide tube 27 is uniform all across the length, but the inner diameter of the guide tube 27 from the back end to the central part in the lengthwise direction thereof is smaller than that of the guide tube 27 from the central part to the distal end in the lengthwise direction. In other words, the wall portion of the guide tube 27 is formed thickly from the back end to the central part and thinly from the central part to the distal end. Thereby, an increased rigidity is imparted to a rear half of the guide tube 27, while a front half of the guide tube 27 is made flexible as compared with the rear half.

With the endoscope cooling device 20E of the present embodiment, flexibility can be imparted to a cover portion of the guide tube 27 and also the rigidity of the guide tube 27 excluding the cover portion can be secured, although it is simple in structure.

In the present embodiment, a thinly formed part functioning as the cover portion 42 is extended from the distal end of the guide tube 27 to the central part in the lengthwise direction. However, there is no particular restriction on a place at which the thinly formed part is provided, and the place may be changed in any way depending on the length of the guide tube 27 or the shape of a test substance. For example, the thin-walled part may be extended from the distal end of the guide tube 27 to the front at the central portion or from the distal end to a part over the central part.

### Sixth Embodiment

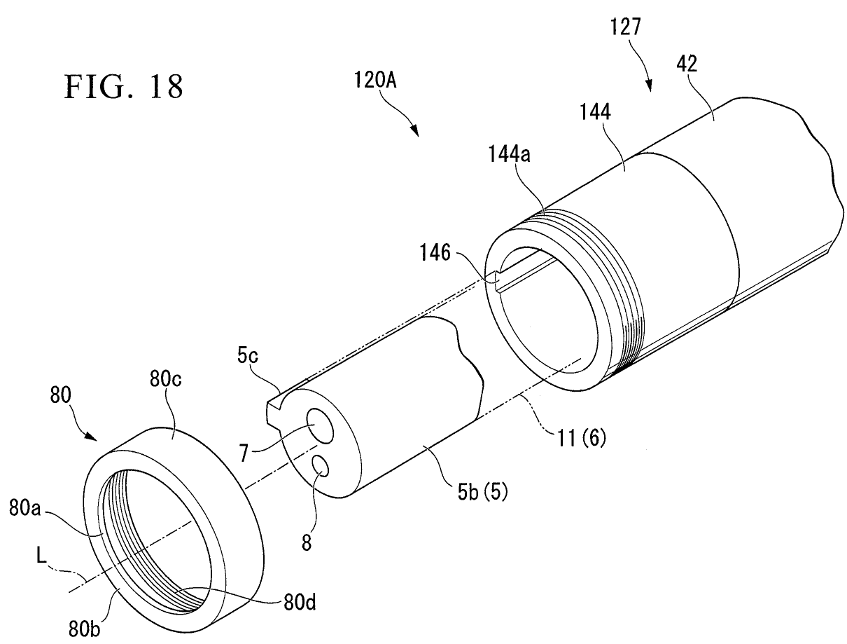

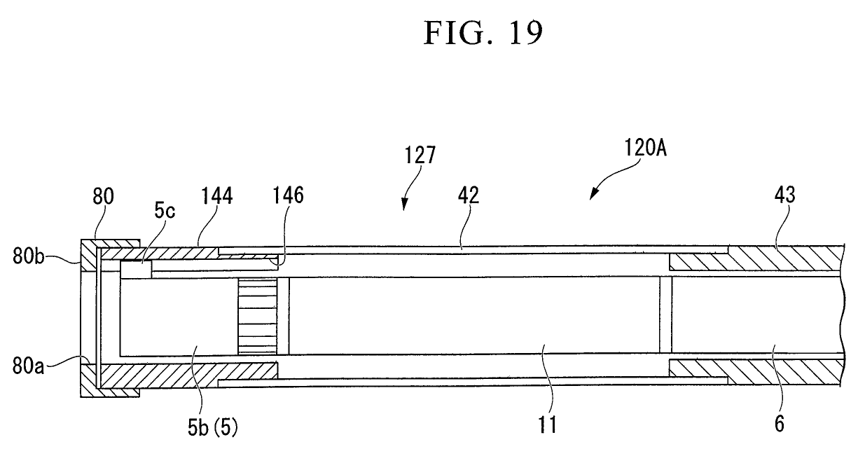

Next, a description is given for a sixth embodiment of the endoscope system of the present invention with reference to FIG. 18 to FIG. 20. In the present embodiment, members common to those used in the above-described embodiment will be given the same reference numerals, the explanation of which will be omitted here.

In the endoscope cooling device 120A of the present embodiment, as shown in FIG. 18 and FIG. 19, a male threaded portion 144a is formed on the outer circumferential face of a distal end base 144. A cap 80 is attached to the male threaded portion 144a of the distal end base 144. The cap 80 includes a main body portion 80b formed approximately in a plate shape having an opening portion 80a and an external fitting portion 80c formed approximately in a cylindrical shape to be projected to the proximal end from the main body portion 80b and fitted at the outside of the distal end base 144. A female threaded portion 80d is formed on the inner circumferential face of the external fitting portion 80c of the cap 80 and screwed onto with the male threaded portion 144a of the distal end base 144.

A groove 146 in parallel with the central axis L in the lengthwise direction of the guide tube 127 is provided inside the distal end base 144. The groove 146 is formed ranging from the distal end face to the back end face of the distal end base 144. Further, a projection 5c to be engaged with the groove 146 is provided on an adaptor 5b to be attached to the distal end portion 5 of the insertion portion 6. Upon attachment of the guide tube 127 to the insertion portion 6, the projection 5c is engaged with the groove 146. The insertion portion 6 is regulated in rotational movement around the central axis L by the projection 5c being engaged with the groove 146 and also able to move back and forth in the lengthwise direction of the guide tube 127 by the projection 5c which is guided to the groove 146. Further, the cap 80 is attached to the distal end base 144. The main body portion 80b of the cap 80 prevents the projection 5c from removing forward from the groove 146.

Next, a description is given for actions of the endoscope 1 and the endoscope cooling device 120A which constitute the endoscope system.

In order to attach the guide tube 127 of the present embodiment to the insertion portion 6, the insertion portion 6 free of the adaptor 5b at the distal end is first inserted into the back end of the guide tube 127 and allowed to project from the distal end of the guide tube 127. After the adaptor 5b is attached to the distal end portion 5 of the insertion portion 6, the insertion portion 6 is drawn into the guide tube 127 to engage the projection 5c with the groove 146. Next, the cap 80 is attached to the distal end base 144. Thereafter, the proximal end of the guide tube 127 is fixed to the insertion portion 6.

In the endoscope cooling device 120A of the present embodiment, cooling air flows through a clearance formed between the insertion portion 6 and the guide tube 127 and is discharged forward from the distal end base 144. At this time, the distal end portion 5 of the insertion portion 6, which is inserted into the guide tube 127, is cooled to protect the observation portion 7 and the illumination portion 8.

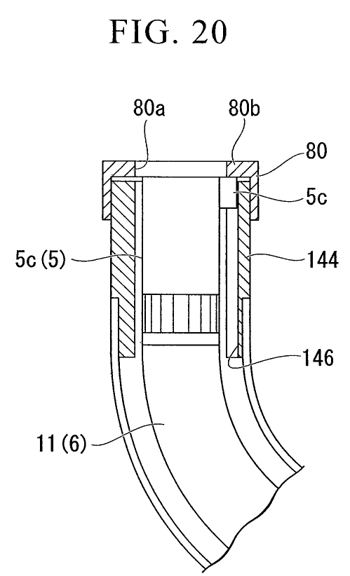

With the endoscope cooling device 120A of the present embodiment, flexibility can be imparted to a cover portion of the guide tube 27 and also rigidity of the guide tube 27 excluding the cover portion can be secured, although it is simple in structure. Further, since the insertion portion 6 is regulated in rotational movement around the central axis L, it is possible to prevent the observation state from changing due to rotation of an image taken by the observation portion 7 through the opening portion 80a of the cap 80. Still further, the main body portion 80b of the cap 80 prevents the projection 5c from removing forward from the groove 146. Therefore, as shown in FIG. 20, even if the insertion portion 6 moves forward relative to the guide tube 127 when the bending portion 11 of the insertion portion 6 is bent, the projection 5c butts against the main body portion 80b. Therefore, there is no chance that the distal end portion 5 of the insertion portion 6 will project from the distal end of the guide tube 127. In other words, it is possible to always keep the distal end portion 5 of the insertion portion 6 inside the guide tube 127.

Further, since the projection 5c is able to move back and forth in the lengthwise direction of the guide tube 127 with respect to the groove 146, the distal end bending portion of the insertion portion 60 can be smoothly moved when it is bent.

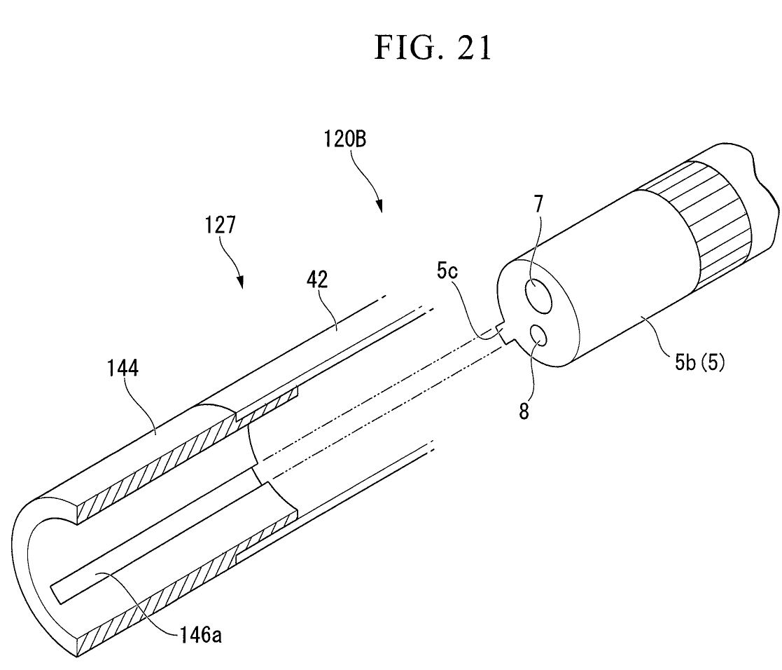

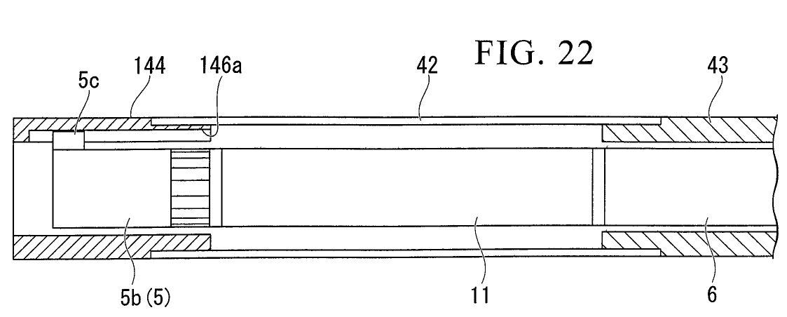

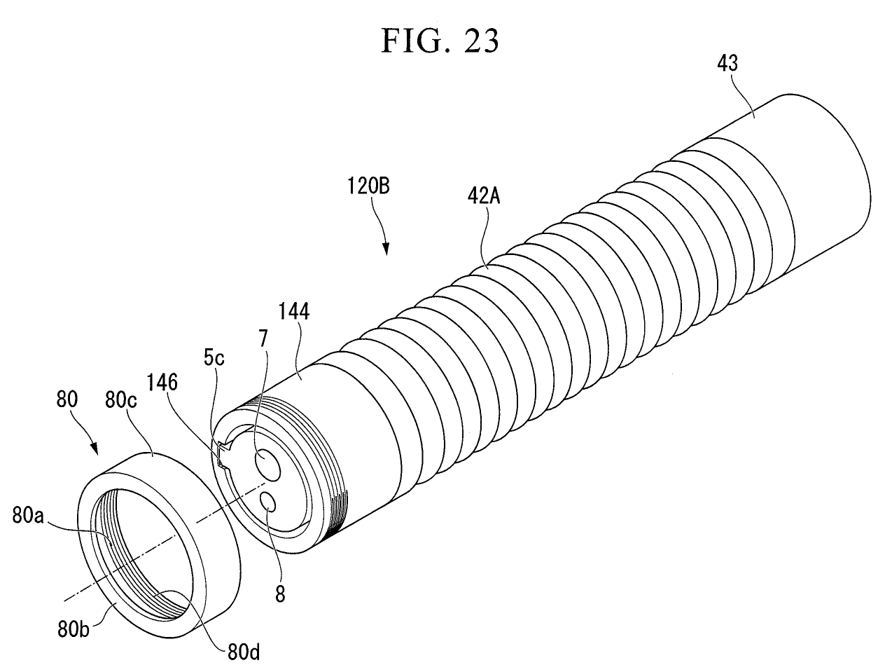

FIG. 21 and FIG. 22 show a first exemplified variation of the present embodiment. In the endoscope cooling device 120B of the exemplified variation, the groove 146a of the distal end base 144 does not reach the distal end face of the distal end base 144. In other words, the groove 146a itself is given a configuration for preventing the projection 5c from removing forward. Therefore, no male threaded portion 144a is formed on the distal end base 144 or no cap 80 is attached thereto.

In order to attach the guide tube 127 of the present embodiment to the insertion portion 6, the insertion portion 6 having the adaptor 5b attached to the distal end is first inserted into the back end of the guide tube 127. In the course of pressing forward the insertion portion 6, the projection 5c is engaged with the groove 146a, and the insertion portion 6 is further pressed forward until the projection 5c butts against the end of the groove 146a. Thereafter, the proximal end of the guide tube 127 is fixed to the insertion portion 6.

The endoscope cooling device 120B of the present exemplified variation also provides the same effects as those described above. In the present embodiment, one projection 5c is provided at the distal end portion 5 of the insertion portion 6. There is no restriction on the number of projections 5c. It is, however, necessary to change the number of the grooves 146 or the arrangement thereof, depending on the change in the number of the projections 5c.

FIG. 23 shows a second exemplified variation of the present embodiment. In the endoscope cooling device 120X of the present exemplified variation, a bellows-like cover portion 42A is adopted to impart a higher flexibility to the guide tube 127. The endoscope cooling device 120B of the present exemplified variation also provides the same effects as those described above.

### Seventh Embodiment

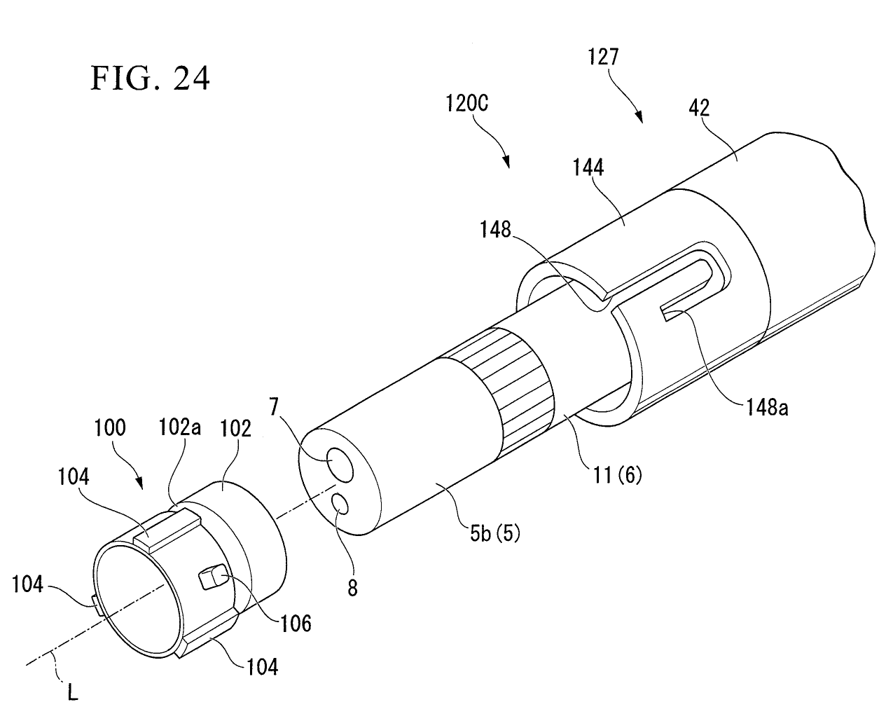

Next, a description is given for a seventh embodiment of the endoscope system of the present invention with reference to FIG. 24. In the present embodiment, members common to those used in the above-described embodiment will be given the same reference numerals, the explanation of which will be omitted here.

As shown in FIG. 24, the endoscope cooling device 120C of the present embodiment is provided with a cylindrical insertion-portion fixing bracket 100 fitted at the outside of the distal end portion 5 of the insertion portion 6. The insertion-portion fixing bracket 100 is provided with a cylindrical main body portion 102, three projected streaks 104 provided on the main body portion 102, and a projection 106 provided on the main body portion 102 separate from the projected streaks 104. The insertion-portion fixing bracket 100 is made of a material such as metal, silicon, polyimide or Teflon. A diameter-reduction portion 102a, which is slightly reduced in inner diameter, is formed at an intermediate portion in the lengthwise direction of the main body portion 102. The inner diameter of the diameter-reduction portion 102a is slightly smaller than the outer diameter of the adaptor 5b. The adaptor 5b is pressed into the main body portion 102 so as to elastically expand the diameter of the diameter reduced portion 102a, by which the insertion-portion fixing bracket 100 is fitted at the outside of the distal end portion 5 of the insertion portion 6. These three projected streaks 104 are provided so as to be spaced at equal intervals in the circumferential direction on the outer circumferential face at the distal end of the main body portion 102, and extended from the distal end up to the intermediate portion in the lengthwise direction of the main body portion 102. The projection 106 is formed between two projected streaks so as to project from the outer circumferential face of the main body portion 102. On the other hand, a slit 148 is formed at the wall portion of the distal end base 144. The slit 148 is formed so as to be cut into the central axis L from the distal end face of the distal end base 144 in parallel and folded back halfway, giving a so-called J letter shape. When the guide tube 127 is attached to the insertion portion 6, the projection 106 is engaged with the deepest portion 148a of the slit 148. The projection 106 is engaged with the slit 148, by which the insertion portion 6 is regulated in rotational movement around the central axis L and is also able to move back and forth in the lengthwise direction of the guide tube 127 by the projection 106 which is guided into the slit 148. Further, since the top faces of the three projected streaks 104 are in contact with the inner circumferential face of the distal end base 144, the distal end portion 5 of the insertion portion 6 is always arranged at the center of the guide tube 127.

Next, a description is given for actions of the endoscope 1 and the endoscope cooling device 120C which constitute the endoscope system.

In order to attach the guide tube 127 of the present embodiment to the insertion portion 6, the insertion portion 6 having the adaptor 5b at the distal end is first inserted into the back end of the guide tube 127 and allowed to project from the distal end of the guide tube 127. After the insertion-portion fixing bracket 100 is fitted at the outside of the distal end portion 5 of the insertion portion 6, the insertion portion 6 is drawn into the guide tube 127 to fit the projection 106 into the slit 148. While the projection 106 is allowed to move along the slit 148, the insertion portion 6 is drawn into the guide tube 127. When the projection 106 butts against a folded portion of the slit 148, the insertion portion 6 is allowed to move backward with respect to the guide tube 127, and the projection 106 is arranged at the deepest portion of the slit 148. Thereafter, the proximal end of the guide tube 127 is fixed to the insertion portion 6.

In the endoscope cooling device 120C of the present embodiment, as with the above-described embodiments, the insertion portion 6 is regulated in rotational movement around the central axis L. Thereby, it is possible to prevent the observation state from changing due to rotation of an image taken by the observation portion 7 via an opening portion of the distal end base 144. Further, since the projection 106 is engaged with the deepest portion of the slit 148, there is no chance that the distal end portion 5 of the insertion portion 6 is projected from the distal end of the guide tube 127. In other words, it is possible to always keep the distal end portion 5 of the insertion portion 6 inside the guide tube 127. In the present embodiment, one projection 106 is provided at the insertion-portion fixing bracket 100. There is no restriction on the number of the projections 106. However, it is necessary to change the number of slits 148 and the arrangement thereof, depending on the number of the projections 106.

### Eighth Embodiment

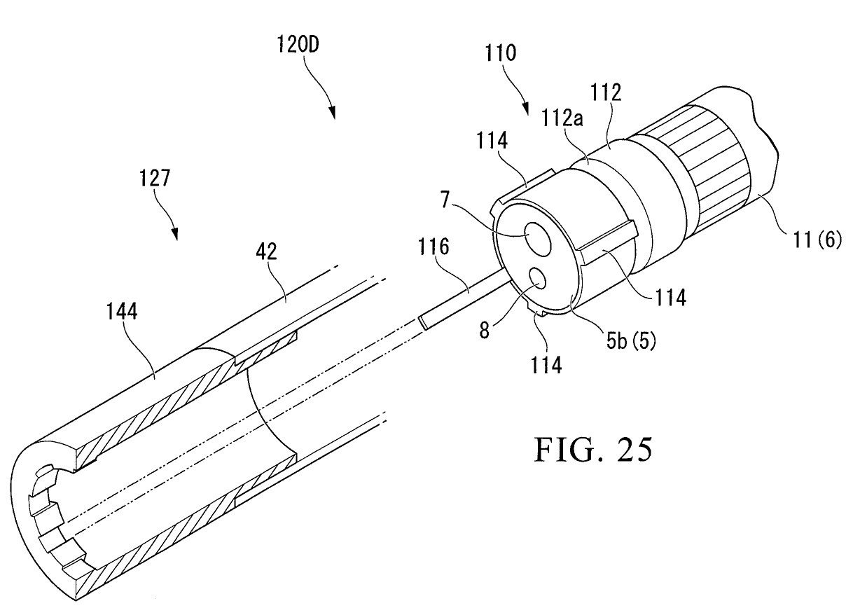

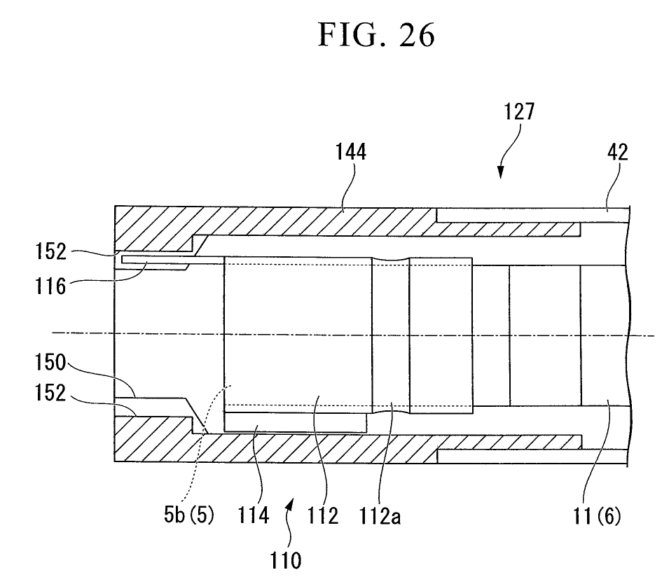

Next, a description is given for an eighth embodiment of the endoscope system of the present invention with reference to FIG. 25 and FIG. 26. In the present embodiment, members common to those used in the above-described embodiment will be given the same reference numerals, the explanation of which will be omitted here.

As shown in FIG. 25 and FIG. 26, the endoscope cooling device 120D of the present embodiment is provided with a cylindrical insertion-portion fixing bracket 110 fitted at the outside of the distal end portion 5 of the insertion portion 6. The insertion-portion fixing bracket 110 is provided with a cylindrical main body portion 112, three projected streaks 114 provided on the main body portion 112 and a bar-like portion 116 provided at the main body portion 112 separately from the projected streaks 114. The insertion-portion fixing bracket 110 is made of a material such as metal, silicon, polyimide or Teflon. A diameter-reduction portion 112a is formed at the main body portion 112. The adaptor 5b is pressed into the main body portion 112, by which the insertion-portion fixing bracket 110 is fitted at the outside of the distal end portion 5 of the insertion portion 6. These three projected streaks 114 are provided on the outer circumferential face at the distal end of the main body portion 112 so as to be spaced at equal intervals in the circumferential direction. The bar-like portion 116 is formed so as to project from the outer circumferential face of the main body portion 102 between two projected streaks formed so as to project forward from the distal end face of the insertion-portion fixing bracket 110. On the other hand, an opening portion 150 reduced in diameter is formed at the distal end of the distal end base 144. The diameter of the opening portion 150 is smaller than the outer diameter of the distal end portion 5 of the insertion portion 6. A plurality of grooves 152 are provided on the inner circumferential face of the opening portion 150. Each of the grooves 152 is formed so as to be in parallel with the central axis L and also spaced at equal intervals in the circumferential direction. The width of the groove 152 is slightly smaller than that of the bar-like portion 116, and the bar-like portion 116 is engaged with any one of the plurality of grooves 152. Therefore, the insertion portion 6 is regulated in rotational movement around the central axis L by the bar-like portion 116 being engaged with the groove 152 and also able to move back and forth in the direction of the central axis L by the bar-like portion 116 being guided into the groove 152. Further, the top faces of the three projected streaks 114 are in contact with the inner circumferential face of the distal end base 144, by which the distal end portion 5 of the insertion portion 6 is always arranged at the center of the guide tube 127.

Next, a description is given for actions of the endoscope 1 and the endoscope cooling device 120D which constitute the endoscope system.

In order to attach the guide tube 127 of the present embodiment to the insertion portion 6, the insertion-portion fixing bracket 100 is first fitted at the outside of the insertion portion 6 having the adaptor 5b attached to the distal end, and the insertion portion 6 is inserted into the back end of the guide tube 127. In the course of pressing forward the insertion portion 6, the bar-like portion 116 is inserted into any one of the grooves 152, and the insertion portion 6 is further pressed forward until the distal end of the bar-like portion 116 reaches the distal end of the guide tube 127. Thereafter, the proximal end of the guide tube 127 is fixed to the insertion portion 6.

In the endoscope cooling device 120D of the present embodiment, cooling air flows through a clearance formed between the insertion portion 6 and the guide tube 127, and is discharged forward from the opening portion 150 of the distal end base 144.

At this time, the distal end portion 5 of the insertion portion 6 inserted into the guide tube 127 is cooled to protect the observation portion 7 and the illumination portion 8. Even if the distal end portion 5 of the insertion portion 6 moves forward with respect to the guide tube 127 and the edge face of the distal end portion 5 is brought closer to the opening portion 150, cooling air is discharged through the groove 152 provided around the opening portion 150. Therefore, there is no chance that the cooling air remains inside the guide tube 127.

According to the endoscope cooling device 120D of the present embodiment, as with the above-described embodiments, the insertion portion 6 is regulated in rotational movement around the central axis L. Therefore, it is possible to prevent the observation state from changing due to rotation of an image taken by the observation portion 7 through an opening portion of the distal end base 144. Further, since the diameter of the opening portion 150 is smaller than the outer diameter of the distal end portion 5 of the insertion portion 6, there is no chance that the distal end portion 5 of the insertion portion 6 is projected from the distal end of the guide tube 127. In other words, the distal end portion 5 of the insertion portion 6 is allowed to remain within the guide tube 127. In the present embodiment, one bar-like portion 116 is provided on the insertion-portion fixing bracket 100. There is no restriction on the number of the bar-like portions 116. It does, however, require special attention, because an excess number of the bar-like portions 116 may inhibit the flow of cooling air leading to the groove 152.

### Ninth Embodiment

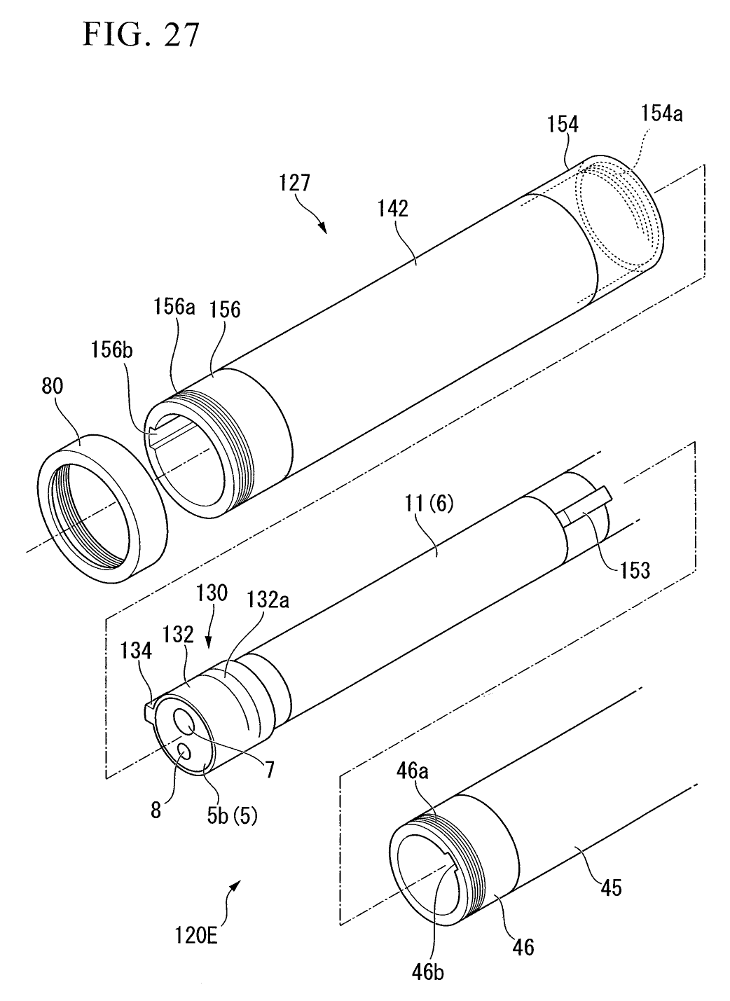

Next, a description is given for a ninth embodiment of the endoscope system of the present invention with reference to FIG. 27. In the present embodiment, members common to those used in the above-described embodiment will be given the same reference numerals, the explanation of which will be omitted here.

As shown in FIG. 27, the endoscope cooling device 120E of the present embodiment is provided with a cylindrical insertion-portion fixing bracket 130 fitted at the outside of the distal end portion 5 of the insertion portion 6. The insertion-portion fixing bracket 130 is provided with a cylindrical main body portion 132 and a projection 134 provided at the main body portion 132. The insertion-portion fixing bracket 130 is made of a material such as metal, silicon, polyimide or Teflon. A diameter-reduction portion 132a is formed at the main body portion 132. The adaptor 5b is pressed into the main body portion 132, by which the insertion-portion fixing bracket 130 is fitted at the outside of the distal end portion 5 of the insertion portion 6. The projection 134 is formed so as to project outwardly in the radial direction from the outer circumferential face of the insertion-portion fixing bracket 130. In addition, a projection 153 is provided at the insertion portion 6 behind the bending portion 11. The projection 153 is also formed so as to project outwardly in the radial direction from the outer circumferential face of the insertion portion 6.

On the other hand, the guide tube 127 is provided with a tube main body 45 made of a rigid material and a cover portion 142 made of a material more flexible than the tube main body 45. A main body base 46 is provided at the distal end of the tube main body 45, and a male threaded portion 46a is formed on the outer circumferential face of the main body base 46. A groove 46b parallel with the length direction of the tube main body 45 is formed on the inner circumferential face of the main body base 46. A proximal end base 154 is provided at the proximal end of the cover portion 142, and a distal end base 156 is provided at the distal end of the cover portion 142. A female threaded portion 154a, which is screwed with the male threaded portion 46a of the main body base 46, is formed on the inner circumferential face of the proximal end base 154. A male threaded portion 156a, which is screwed into the cap 80, is formed on the outer circumferential face of the distal end base 156. A groove 156b parallel with the length direction of the cover portion 142 is formed on the inner circumferential face of the distal end base 156. A projection 153 provided behind the cover portion 142 is engaged with the groove 46b, and a projection 134 provided at the distal end portion 5 of the insertion portion 6 is engaged with the groove 156b. Therefore, the projection 153 is engaged with the groove 46b and the projection 134 is also engaged with the groove 156b, by which the insertion portion 6 is regulated in rotational movement around the central axis L. At the same time, the projection 153 is guided into the groove 46b and the projection 134 is guided into the groove 156b, by which the insertion portion 6 is able to move back and forth in the direction of the central axis L. Further, the cap 80 is attached to the distal end base 156, thereby the main body portion 80b of the cap 80 prevents the projection 134 from being removed forward from the groove 156b.

Next, a description is given for actions of the endoscope 1 and the endoscope cooling device 120E which constitute the endoscope system.

In order to attach the guide tube 127 of the present embodiment to the insertion portion 6, the female threaded portion 154a of the proximal end base 154 is first screwed onto the male threaded portion 46a of the main body base 46, and after the cover portion 142 is attached to the distal end of the tube main body 45, the insertion portion 6 is inserted into the back end of the guide tube 127 and projected from the distal end of the guide tube 127. The insertion-portion fixing bracket 130 is fitted at the outside of the distal end portion 5 of the insertion portion 6, and the insertion portion 6 is drawn into the guide tube 127. Next, the projection 153 is engaged with the groove 46b and the projection 134 is also engaged with the groove 156b. Then, the cap 80 is attached to the distal end base 156. Thereafter, the proximal end of the guide tube 127 (tube main body 45) is fixed to the insertion portion 6.

In the endoscope cooling device 120E of the present embodiment, the insertion portion 6 is regulated in rotational movement around the central axis L at two points before and after the bending portion 11. Therefore, it is possible to more securely prevent the observation state from changing due to rotation of an image taken by the observation portion 7 through an opening portion of the distal end base 144. Further, the cap 80 is prevented by the main body portion 80b from being removed forward from the groove 156b of the projection 134. In other words, it is possible to always keep the distal end portion 5 of the insertion portion 6 inside the guide tube 127. In the present embodiment, the projections 134 and 153 are provided respectively by one unit. There is no restriction on the number of the projections 134, 153. However, it is necessary to change the number of the grooves 46b, 156b and the arrangement thereof, depending on the number of the projections 134, 153.

### Tenth Embodiment

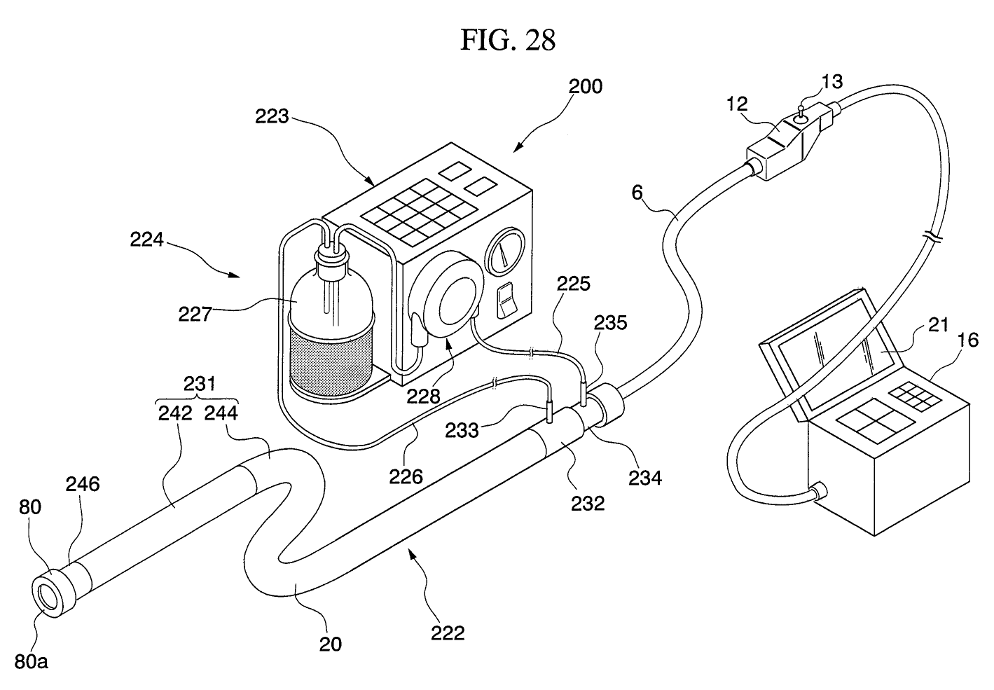

Next, a description is given for a tenth embodiment of the endoscope system of the present invention with reference to FIG. 28 to FIG. 30. In the present embodiment, members common to those used in the above-described embodiment will be given the same reference numerals, the explanation of which will be omitted here.

As shown in FIG. 28, the endoscope system of the present embodiment is provided with a direct-viewing-type endoscope 1 and an endoscope cooling device 200 for allowing a cooling fluid such as air and water to flow at the insertion portion 6 of the endoscope 1, thereby cooling the distal end of the insertion portion 6.

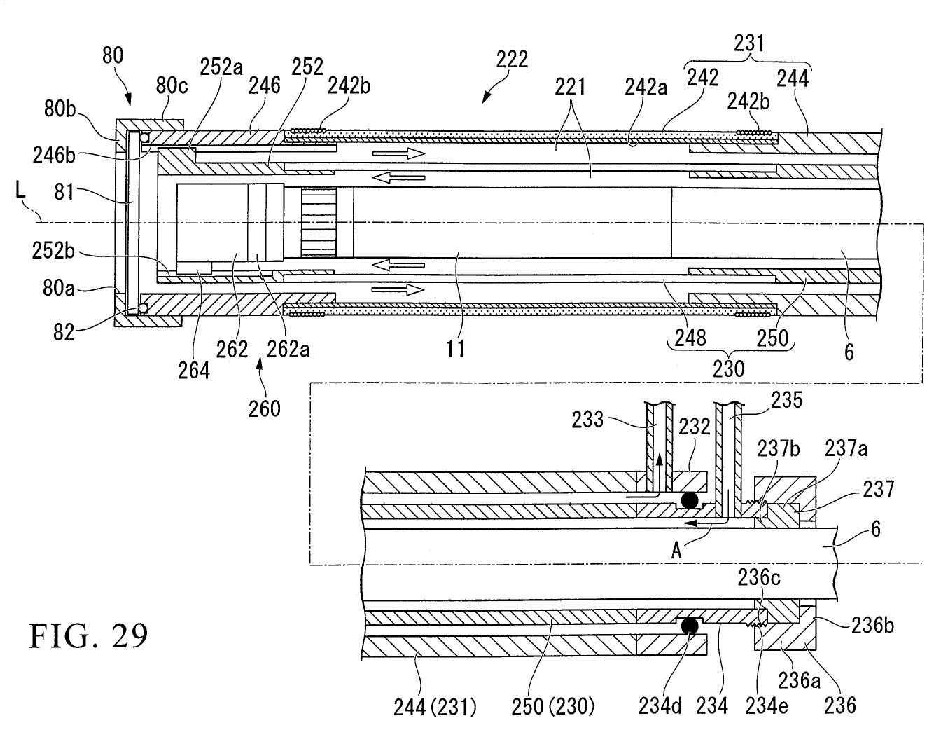

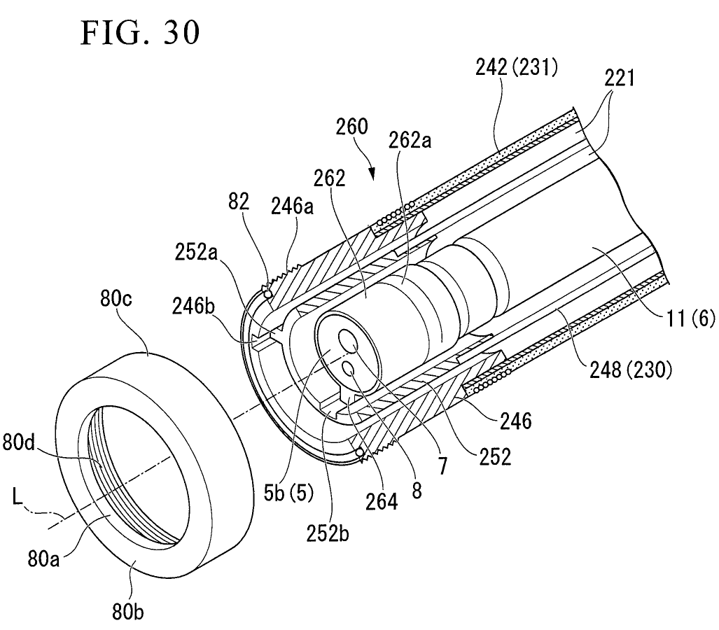

As shown in FIG. 29 and FIG. 30, the endoscope cooling device 200 is provided with a guide tube 222 attached to the distal end of the insertion portion 6 so as to form a cooling flow path 221 through which a cooling fluid flows between the outer circumferential face of the insertion portion 6 and the guide tube and a fluid flowing portion 223 for supplying the cooling fluid to a cooling flow path 221 and recovering the fluid. The fluid flowing portion 223 is provided with a cooling-fluid supply source 224, a supply pipe 225 for supplying the cooling fluid from the supply source 224 to the guide tube 222, and a discharge pipe 226 for recovering the cooling fluid into the supply source 224. These portions are connected to the guide tube 222 via connecting joints 235, 233 to be described later. Further, the supply source 224 is provided with a compressor 227 for reserving the cooling fluid and a pump 228 for supplying the cooling fluid inside the compressor 227 to the supply pipe 225.

The guide tube 222 is provided with an outer sheath 231 and an inner sheath 230 inserted inside the outer sheath 231. The outer sheath 231 is made of a flexible material, for example, a resin material such as a foamed fluorine tube, and provided with a cover portion 242 for covering the bending portion 11 of the insertion portion 6 and an outer sheath main body 244 made of a material more rigid than the cover portion 242, for example, a fluorine tube, to cover the insertion portion 6 at the rear of the bending portion 11.

A flexible and thin tube 242a made of silicon, etc., for preventing the leakage of a fluid is arranged inside the cover portion 242. An outer sheath base 246 is provided at the distal end of the outer sheath 231. The outer sheath base 246 is tied to the distal end of the outer sheath 231 by using a thread-like member 242b, and the outer sheath main body 244 is also tied to the proximal end of the outer sheath 230 by using the thread-like member 242b. A male threaded portion 246a, which is screwed into the cap 80, is formed on the outer circumferential face of the outer sheath base 246. A groove 246b in parallel with the length direction of the cover portion 242 is formed on the inner circumferential face of the outer sheath base 246. The inner sheath 230 is made of a flexible material, for example, a resin such as a foam fluorine tube, and provided with a cover portion 248 for covering the bending portion 112 of the insertion portion 6 and an inner sheath main body 250 made of a material more rigid than the cover portion 248, such as a fluorine tube, to cover the insertion portion 6 at the rear of the bending portion 11. An inner sheath base 252 is provided at the distal end of the inner sheath 230. A projection 252a projected outward in the radial direction is formed on the outer circumferential face of the inner sheath base 252, and a groove 252b parallel with the length direction of the cover portion 248 is formed on the inner circumferential face of the inner sheath base 252. The projection 252a of the inner sheath base 252 is engaged with the groove 246b of the outer sheath base 246.

On the other hand, a cylindrical insertion-portion fixing bracket 260 is fitted into the distal end portion 5 of the insertion portion 6. The insertion-portion fixing bracket 260 is provided with a cylindrical main body portion 262 and a projection 264 provided at the main body portion 262. The insertion-portion fixing bracket 260 is made of a material such as metal, silicon, polyimide or Teflon. A diameter-reduction portion 262a is formed at the main body portion 262. The adaptor 5b is pressed into the main body portion 262, by which the insertion-portion fixing bracket 260 is fitted at the outside of the distal end portion 5 of the insertion portion 6. The projection 264 is formed so as to project outwardly in the radial direction from the outer circumferential face of the insertion-portion fixing bracket 260 and engaged with the groove 252b of the inner sheath base 252. The projection 252a is engaged with the groove 246b and the projection 264 is also engaged with the groove 252b, by which the insertion portion 6 is regulated in rotational movement around the central axis L. At the same time, the projection 252a is guided into the groove 246b and the projection 264 is also guided into the groove 252b, by which the insertion portion 6 is able to move back and forth in the lengthwise direction of the guide tube 222. Therefore, the bending movement can be made smoothly. Further, the cap 80 is attached to the outer sheath base 246 via the cover glass 81. The cap 80 prevents the projection 252a from removing forward from the groove 246b. In addition, the cap 80 prevents the projection 264 from removing forward from the groove 252b.

A proximal end outer base 232 is connected to the proximal end of the outer sheath main body 244. A connecting joint 233 connected to a discharge pipe 226 of the fluid flowing portion 223 is provided at the proximal end outer base 232.

A proximal end inner base 234 is connected to the proximal end of the inner sheath main body 250. An annular groove is formed on the outer circumferential face of the proximal end inner base 234, and an O-ring 234d is fitted at the outside of the groove. The O-ring 234d seals a space between the outer circumferential face of the proximal end inner base 234 and the inner circumferential face of the proximal end outer base 232. Further, a connecting joint 235 connected to a supply pipe 225 of the fluid flowing portion 223 is provided at the proximal end inner base 234.

A male threaded portion 234e is formed on the proximal end outer circumferential face of the proximal end inner base 234, and a fixing member 236 is screwed onto the male threaded portion 234e. The fixing member 236 is provided with an approximately cylindrical main body portion 236a and an inner flange portion 236b projected to the inner circumferential face at the proximal end of the main body portion 236a. A female threaded portion 236c, which is screwed onto the male threaded portion 234e of the proximal end inner base 234, is formed on the proximal-end inner circumferential face at the main body portion 236a of the fixing member 236. Further, the inner diameter of the inner flange portion 236b of the fixing member 236 is set to be slightly larger than the outer diameter of the insertion portion 6, into which the proximal end of the insertion portion 6 which is attached to the inner sheath 230 is inserted.

A seal member 237 is set between the proximal end of the proximal end inner base 234 and the inner flange portion 236b of the fixing member 236 inside the main body portion 236a of the fixing member 236. The seal member 237 is an approximately tubular member made of an elastically deformable material such as rubber and provided with a main body portion 237a held between the proximal end of the proximal end inner base 234 and the inner flange portion 236b of the fixing member 236 and a fitting portion 237b which is fitted into the inner circumferential face of the proximal end inner base 234, with the outer diameter being reduced from the main body portion 237a. The outer diameter of the main body portion 237a of the seal member 237 is set to be approximately equal to the inner diameter of the main body portion 236a of the fixing member 236. Further, the inner diameter of the main body portion 237a of the seal member 237 and that of the fitting portion 237b are both set to be approximately equal to the outer diameter of the insertion portion 6 which is to be inserted. Therefore, the seal member 237 held therebetween swells out to the inner circumferential face and the outer circumferential face by tightening the fixing member 236 with the proximal end inner base 234, in a state in which the insertion portion 6 is attached. Thereby, the insertion portion 6 is fixed by the seal member 237 to seal a space between the insertion portion 6 and the inner sheath 230.

Next, a description is given for actions of the endoscope 1 and the endoscope cooling device 200 which constitute the endoscope system.

In order to attach the guide tube 222 of the present embodiment to the insertion portion 6, the insertion portion 6 having an adaptor 5b attached at the distal end is first inserted into the back end of the guide tube 222 and projected from the distal end of the guide tube 222. After the insertion-portion fixing bracket 260 is fitted at the outside of the distal end portion 5 of the insertion portion 6, the insertion portion 6 is drawn into the guide tube 222, thereby engaging the projection 264 with the groove 252b. Next, the cap 80 is attached to the outer sheath base 246. Thereafter, the proximal end of the guide tube 222 is fixed to the insertion portion 6.

In the endoscope cooling device 200 of the present embodiment, cooling air supplied from the fluid flowing portion 223 via the supply pipe 225 and the connecting joint 235 flows forward through a clearance formed between the insertion portion 6 and the inner sheath 230 and is ejected from the distal end of the inner sheath base 252 to a cover glass 81. The cooling air ejected from the inner sheath base 252 flows backward through a clearance formed between the inner sheath 230 and the outer sheath 231, and is discharged to the compressor 227 via the connecting joint 233 and the discharge pipe 226. At this time, the distal end portion 5 of the insertion portion 6 which is inserted into the guide tube 222 is cooled to protect the observation portion 7 and the illumination portion 8.

According to the endoscope cooling device 200 of the present embodiment, as with the above-described embodiment, the insertion portion 6 is regulated in rotational movement around the central axis L. It is, therefore, possible to prevent the observation state from changing due to rotation of an image taken by the observation portion 7 via the cover glass 81 of the cap 80.

### Eleventh Embodiment

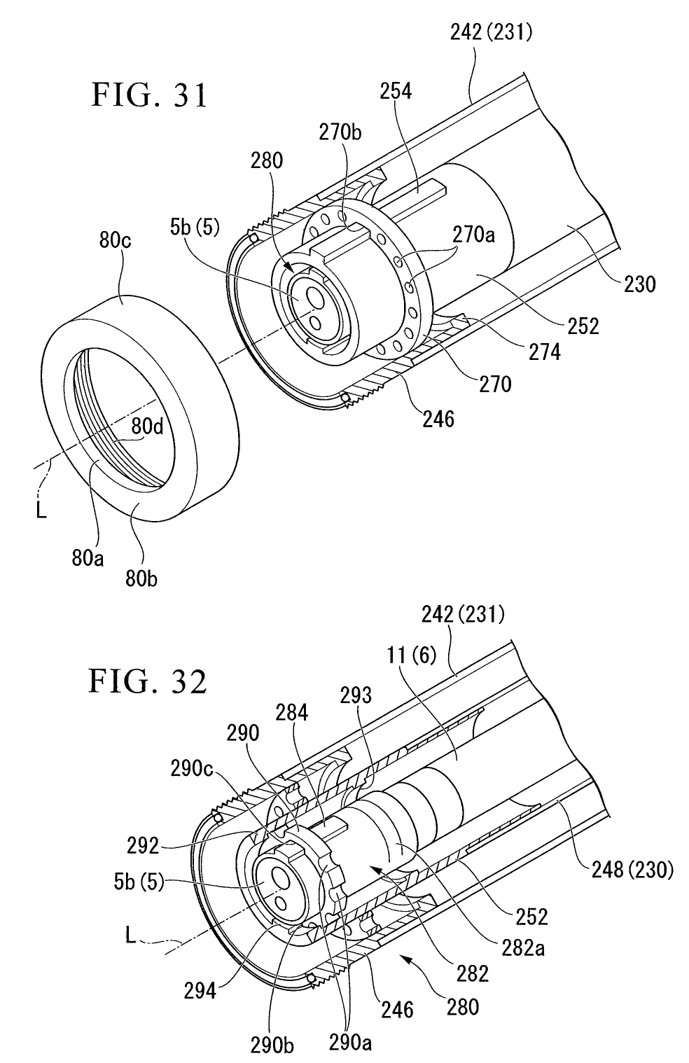

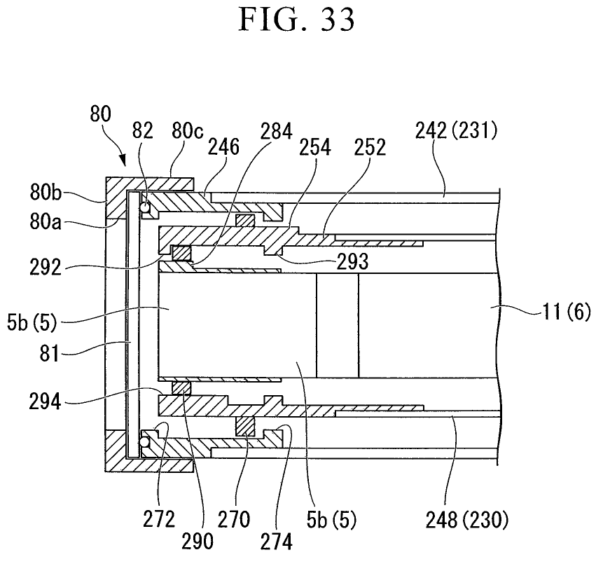

Next, a description is given for an eleventh embodiment of the endoscope system of the present invention with reference to FIG. 31 to FIG. 33. In the present embodiment, members common to those used in the above-described embodiment will be given the same reference numerals, the explanation of which will be omitted here.

In the endoscope cooling device 210 of the present embodiment, as shown in FIG. 31 to FIG. 33, an outer ring member 270 is set between the outer sheath base 246 and the inner sheath base 252. A rib 274 for regulating a movable range of the outer ring member 270 in the lengthwise direction of the guide tube 222 is formed at the back end of the inner circumferential face of the outer sheath base 246. A plurality of through holes 270a penetrating through in the lengthwise direction of the guide tube 222 are formed at the outer ring member 270. Further, a notch 270b is formed on the inner circumferential face of the outer ring member 270. On the other hand, a projected streak 254 is formed along the length direction of the guide tube 222 on the outer circumferential face of the inner sheath base 252. The projected streak 254 is engaged with the notch 270b of the outer ring member 270, and the outer ring member 270 is regulated in moving in the circumferential direction with respect to the inner sheath base 252.

A cylindrical insertion-portion fixing bracket 280 is fitted at the outside of the distal end portion 5 of the insertion portion 6. The insertion-portion fixing bracket 280 is provided with a cylindrical main body portion 282 and a projected streak 284 provided on the outer circumferential face of the main body portion 282. The insertion-portion fixing bracket 280 is made of a material such as metal, silicon, polyimide or Teflon. A diameter-reduction portion 282a is formed at the main body portion 282. The adaptor 5b is pressed into the main body portion 282, by which the insertion-portion fixing bracket 280 is fitted at the outside of the distal end portion 5 of the insertion portion 6. The projected streak 284 is formed along the length direction of the main body portion 282.

An inner ring member 290 is set between the insertion-portion fixing bracket 280 fitted at the outside of the distal end of the insertion portion 6 and the inner sheath base 252. Ribs 292, 293 for regulating the movable range of the inner ring member 290 in the lengthwise direction of the guide tube 222 are formed at the front end and an intermediate portion of the inner circumferential face of the inner sheath base 252. The ribs 292, 293 formed at the front end and the intermediate portion on the inner circumferential face of the inner sheath base 252 are constituted so as to be divided. A plurality of notches 290a constituting a flow path of cooling air is formed on the outer circumferential face of the inner ring member 290. Further, another notch 290b is formed on the outer circumferential face of the inner ring member 290. A similar notch 290c is also formed on the inner circumferential face of the inner ring member 290. A projected streak 294 is formed along the length direction of the guide tube 222 on the inner circumferential face of the inner sheath base 252. The projected streak 294 of the inner sheath base 252 is engaged with the notch 290b of the inner ring member 290 and the projected streak 284 of the insertion-portion fixing bracket 280 is engaged with the notch 290c of the inner ring member 290, by which the ring member 290 is regulated in moving in the circumferential direction with respect to both the inner sheath base 252 and the insertion portion 6.

Next, a description is given for actions of the endoscope 1 and the endoscope cooling device 210 which constitute the endoscope system.

In the endoscope cooling device 210 of the present embodiment, cooling air supplied from the fluid flowing portion 223 via the supply pipe 225 and the connecting joint 235 flows forward through a clearance formed between the insertion portion 6 and the inner sheath 230, passes through the notch 290a of the inner ring member and is ejected from the distal end of the inner sheath base 252 to the cover glass 81. The cooling air ejected from the inner sheath base 252 is blocked by the cover glass 81 and flows in reverse. Next, the cooling air passes through the through hole 270a of the outer ring member 270, flows backward through a clearance formed between the inner sheath 230 and the outer sheath 231 and is discharged to the compressor 227 via the connecting joint 233 and the discharge pipe 226. At this time, the distal end portion 5 of the insertion portion 6 inserted into the guide tube 222 is cooled to protect the observation portion 7 and the illumination portion 8.

According to the endoscope cooling device 210 of the present embodiment, as with the above-described embodiment, the insertion portion 6 is regulated in rotational movement around the central axis L. It is, therefore, possible to prevent the observation state from changing due to rotation of an image taken by the observation portion 7 via the cover glass 81 of the cap 80.

### Twelfth Embodiment

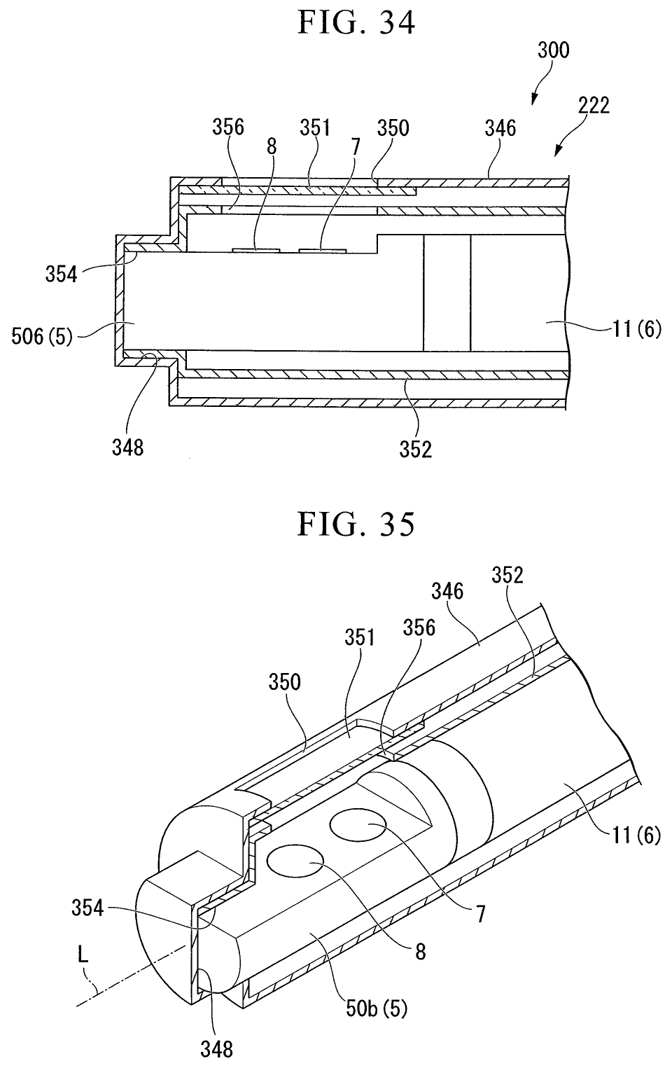

Next, a description is given for a twelfth embodiment of the endoscope system of the present invention with reference to FIG. 34 and FIG. 35. In the present embodiment, members common to those used in the above-described embodiment will be given the same reference numerals, the explanation of which will be omitted here.

As shown in FIG. 34 and FIG. 35, the endoscope used in the present embodiment is a so-called side-view type endoscope. In the side-view type endoscope, the distal end portion 5 of the insertion portion 6 is such that the cross section of which is formed approximately in a semi-circle, with the circle being partially cut out.

In the endoscope cooling device 300 of the present embodiment, the distal end of the inner sheath base 352 is reduced in diameter to form an opening portion 354 which is formed approximately in a semi-circle and engaged so as to slide the adaptor 50b (that is, the distal end portion 5) attached to the distal end of the insertion portion 6. Further, at the inner sheath base 352, an inner window portion 356 for exposing the observation portion 7 and the illumination portion 8 of the side-view type endoscope is formed.

Further, in the endoscope cooling device 300 of the present embodiment, the distal end of the outer sheath base 346 is reduced in diameter to form a recessed portion 348 fitted at the distal end of the inner sheath base 352. An outer window portion 350 for exposing the illumination portion 8 is formed at the outer sheath base 346 so as to overlap with the inner window portion 356. A cover glass 351 is attached to the outer window portion 350 from inside.

The distal end portion 5 of the insertion portion 6 is engaged so as to slide on the opening portion 354 of the inner sheath base 532, and the distal end of the inner sheath base 352 is fitted into the recessed portion 348 of the outer sheath base 346. The shape of the cross section formed approximately in a semi-circle acts as a key, by which the insertion portion 6 is regulated in rotational movement around the central axis L. At the same time, the distal end portion 5 slides on the opening portion 354 of the inner sheath base 352, by which it is able to move back and forth in the direction of the central axis L.

Next, a description is given for actions of the endoscope 1 and the endoscope cooling device 300 which constitute the endoscope system.

In the endoscope cooling device 300 of the present embodiment, cooling air supplied from the fluid flowing portion 223 via the supply pipe 225 and the connecting joint 235 flows forward through a clearance formed between the insertion portion 6 and the inner sheath 230 and flows into the inner window portion 356 by being blocked by a closed front wall of the inner sheath base 352. The cooling air, which has passed through the inner window portion 356, flows backward through a clearance formed between the inner sheath 230 and the outer sheath 231, and is discharged to the compressor 227 via the connecting joint 233 and the discharge pipe 226. At this time, the distal end portion 5 of the insertion portion 6 inserted into the guide tube 222 is cooled to protect the observation portion 7 and the illumination portion 8.

According to the endoscope cooling device 300 of the present embodiment, as with the above-described embodiment, the insertion portion 6 is regulated in rotational movement around the central axis L. It is, therefore, possible to prevent the observation state from changing due to rotation of an image taken by the observation portion 7 via the inner window portion 356 and the outer window portion 350.

### Thirteenth Embodiment

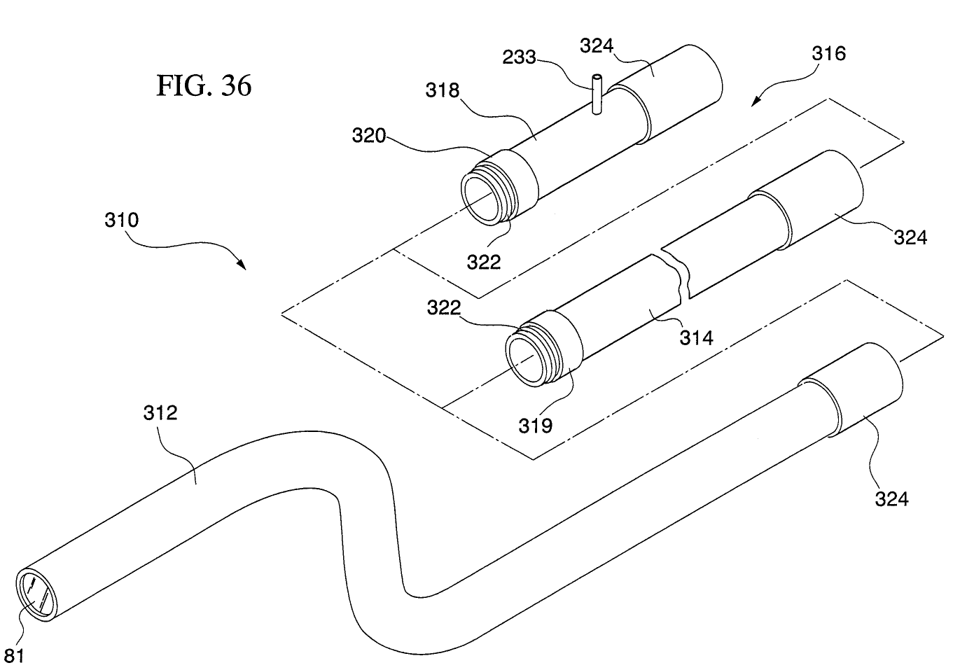

Next, a description is given for a thirteenth embodiment of the endoscope system of the present invention with reference to FIG. 36. In the present embodiment, members common to those used in the above-described embodiment will be given the same reference numerals, the explanation of which will be omitted here.

In the endoscope cooling device 310 of the present embodiment, as shown in FIG. 36, a sheath extending portion 314 communicatively connected to a returning flow path (not illustrated) through which a cooling fluid flows is connected to the proximal end of the outer sheath 312 in a removable manner. The outer sheath 312 is formed shorter in length than the outer sheath 20 of the first embodiment in such a manner that it is to be approximately equal in length to the outer sheath 20 of the first embodiment by the sheath extending portion 314 to the proximal end thereof being attached. The second base 316 is provided with a metal-made base main body 318 and a connector portion 320 attached to the base main body 318 in a removable manner. A packing 322, which can be fitted into the inner face of the base main body 318, is provided at the distal end of the connector portion 320. A fixing portion 324 to an inner sheath (not illustrated) is provided at the proximal end of the connector portion 320.

A packing 322 similar to that provided at the connector portion 320 is provided at the distal end of the sheath extending portion 314, and a base main body 318 similar to that installed in the outer sheath 312 is provided at the proximal end thereof. In other words, the connector portion 320 can be attached in a removable manner both to the base main body 318 of the outer sheath 312 and that of the sheath extending portion 314.

Next, a description is given for actions of the endoscope and the endoscope cooling device 310 which constitute the endoscope system.

Where the insertion portion 6 inserted into a test substance is short, the connector portion 320 is fitted into the base main body 318 provided at the proximal end of the outer sheath 312 and used similarly to the above-described embodiment.

Where the insertion portion 6 inserted into a test substance is long, the base main body 318 provided at the proximal end of the outer sheath 312 is fitted into a packing 322 provided at the distal end of the sheath extending portion 314. Next, the connector portion 320 is fitted into the base main body 318 provided at the proximal end of the sheath extending portion 314, which is used similarly as with the above-described embodiment. At this time, a flow path communicatively connected to a sheath flow path (not illustrated) is formed between the sheath extending portion 314 and the inner sheath. Therefore, a cooling fluid flowing through the insertion portion flow path (not illustrated) flows from the sheath flow path to this flow path and is discharged from a fluid discharge port 233 of the connector portion 320.

According to the endoscope cooling device 310 of the present embodiment, where the insertion portion 6 inserted into a test substance is short, only the outer sheath 312 is attached to the insertion portion 6, or it may be used by adding the sheath extending portion 314, if necessary. Thereby, it is able to provide the sheath with various lengths depending on the application. Further, the sheath is divided into smaller portions so that a deteriorated portion can be exchanged more effectively.

### Fourteenth Embodiment

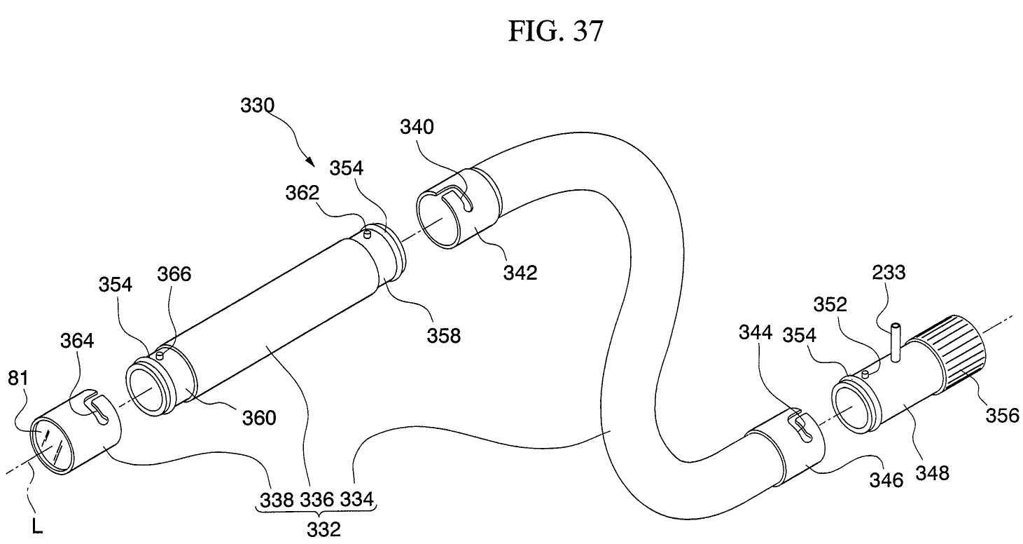

Next, a description is given for a fourteenth embodiment of the endoscope system of the present invention with reference to FIG. 37. In the present embodiment, members common to those used in the above-described embodiment will be given the same reference numerals, the explanation of which will be omitted here.

As shown in FIG. 37, in the endoscope cooling device 330 of the present embodiment, the outer sheath 332 forms a cooling-air flow path between the outer circumferential face of the inner sheath and the outer sheath and is provided with an outer sheath main body 334 positioned in the vicinity of the proximal end of the bending portion, a tubular flexible portion 336 connected to the distal end of the outer sheath main body 334, and a distal end base 338 connected to the distal end of the flexible portion 336 in a removable manner.

A third base 342 having a distal end groove 340 with which the proximal end raised portion 362 of the flexible portion 336 can be engaged is provided at the distal end of the outer sheath main body 334. A base main body 348 of a second base 346 having a proximal end groove 344 with which a distal end raised portion 352 on the connector side can be engaged is provided at the proximal end of the outer sheath main body 334. The distal end raised portion 352 on the connector side, which is engaged with the proximal end groove 344 provided at the base main body 348, and an O-ring 354, which is pressed to the inner face of the base main body 348 at the distal end from the proximal-end raised portion 352 on the connector side, are provided at the distal end of the connector portion 350. A fixing portion 356 to the inner sheath is provided at the proximal end of the connector portion 350.

The flexible portion 336 is made of a material more flexible than the outer sheath main body 334. A proximal end short tube 358 is bonded to the proximal end of the flexible portion 336, while a proximal-end short tube 360 is bonded to the distal end. A proximal end raised portion 362, which is engaged with the distal end groove 340 of a third base 342, and an O-ring 354, which is pressed to the inner face of the third base 342 closer to the proximal end than the proximal end raised portion 362, are provided at the proximal end short tube 358. A distal end raised portion 366, which can be engaged with the distal end base groove 364 provided at the distal end base 338, and the O-ring 354, which is pressed to the inner face of the distal end base 338 closer to the distal end than the distal end raised portion 366, are provided at the proximal-end short tube 360.

The distal end base 338 is formed in a short tube shape, and the cover glass 81 is provided at the distal end. The distal end base groove 364 is provided at the proximal end of the distal end base 338. In addition, the distal end groove 340, the proximal end groove 344 and the distal end base groove 364 are all extended from the base edge portion to the direction of the central axis L and bent in the midstream in a direction orthogonal to the central axis L.

Next, a description is given for actions of the endoscope and the endoscope cooling device 330 which constitute the endoscope system.

First, the distal end base groove 364 of the distal end base 338 is engaged with the proximal-end raised portion 366 of the flexible portion 336, the proximal end raised portion 362 of the flexible portion 336 is engaged with the distal end groove 340 of the third base 342 and also the proximal end groove 344 of the base main body 348 is engaged with the proximal-end raised portion 352 on the connector side at the connector portion 350 to give an outer sheath 332. Next, as with the above-described embodiment, the inner sheath is fixed to the insertion portion 6. In addition, there is no restriction on the order of the above assembly. Thereafter, a cooling fluid is supplied and circulated through a returning flow path (not illustrated).

The endoscope cooling device 330 of the present embodiment is able to provide the same effects as those of the above-described embodiment. In particular, where there is found a necessity for exchanging the flexible portion 336 due to the deterioration, only the flexible portion 336 can be removed from the outer sheath 332 for exchange.

### Fifteenth Embodiment

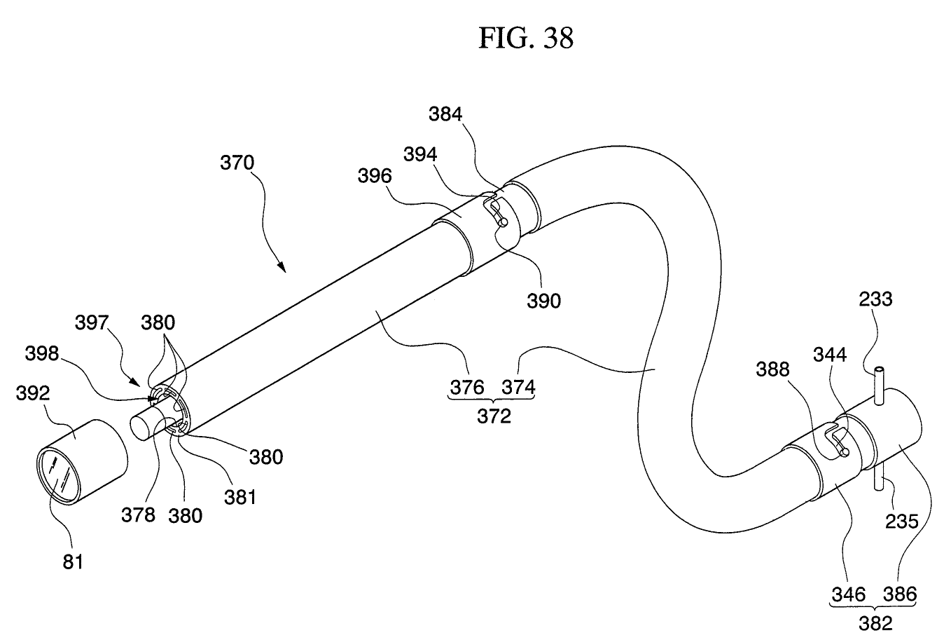

Next, a description is given for a fifteenth embodiment of the endoscope system of the present invention with reference to FIG. 38. In the present embodiment, members common to those used in the above-described embodiment will be given the same reference numerals, the explanation of which will be omitted here.

As shown in FIG. 38, in the endoscope cooling device 370 of the present embodiment, the sheath 372 is provided with a sheath main body 374 and a flexible portion 376 connected to the distal end of the sheath main body 374 in a removable manner. An insertion hole 378 into which the insertion portion 6 can be inserted and a plurality of small flow paths 380 arrayed in the circumferential direction around the insertion hole 378 are respectively arranged at the sheath main body 374 and the flexible portion 376.

The base main body 346 is provided at the proximal end of the sheath main body 374, and a third base 384 is provided at the distal end. A raised portion 388 on the connector side, which can be engaged with the proximal end groove 344 of the base main body 348, and which is formed at the connector portion 386 and the fluid supply port 235, and the fluid discharge port 233 are provided.

A main-body raised portion 390, which can be engaged with a flexible portion groove 394, is provided at the third base 384. A distal end base 392 having the cover glass 81 is bonded to the distal end of the flexible portion 376. A flexible portion base 396 having the groove 394 at the flexible portion, with which the main-body raised portion 390 can be engaged, is provided at the proximal end of the flexible portion 376.

The small flow paths 380 on the sheath main body 374 are provided so as to be opened at the distal end of the sheath main body 374 to form a sheath flow path 397. When the insertion portion 6 is inserted into an insertion hole 378, an insertion-portion flow path 398 is formed between the insertion portion 6 and the wall face of the insertion hole 378. Therefore, the insertion-portion flow path 398 is communicatively connected to the sheath flow path 397 via a space formed between the distal end face 381 of the flexible portion 376 and the cover glass 81.

Next, a description is given for actions of the endoscope and the endoscope cooling device 370 which constitute the endoscope system.

First, the main-body raised portion 390 at the third base 384 of the sheath main body 374 is engaged with the flexible-portion groove 394 at the flexible portion base 396 of the flexible portion 376 to connect the flexible portion 376 with the sheath main body 374. Further, the proximal end groove 344 at the base main body 348 of the sheath main body 374 is engaged with the raised portion 388 on the connector side at the connector portion 386 to connect the sheath main body 374 with the connector portion 386, thereby forming the sheath 372. Next, the insertion hole 378 and the insertion portion 6 are fixed in such a manner that the distal end of the insertion portion 6 is separated from the cover glass 81 only by a predetermined distance. At this time, an insertion-portion flow path 398 is formed between the insertion portion 6 and the wall face of the insertion hole 378. Next, the insertion portion 6 is inserted into a test substance, together with the sheath 372, thereby supplying a cooling fluid to the insertion-portion flow path 398. The cooling fluid flows through the insertion- portion flow path 398 and is returned inside the distal end base 392. Next, it flows inside the small flow paths 380 on the sheath main body 374 for circulation. The endoscope cooling device 370 of the present embodiment is able to provide the same effects as those of the above embodiment.

### Sixteenth Embodiment

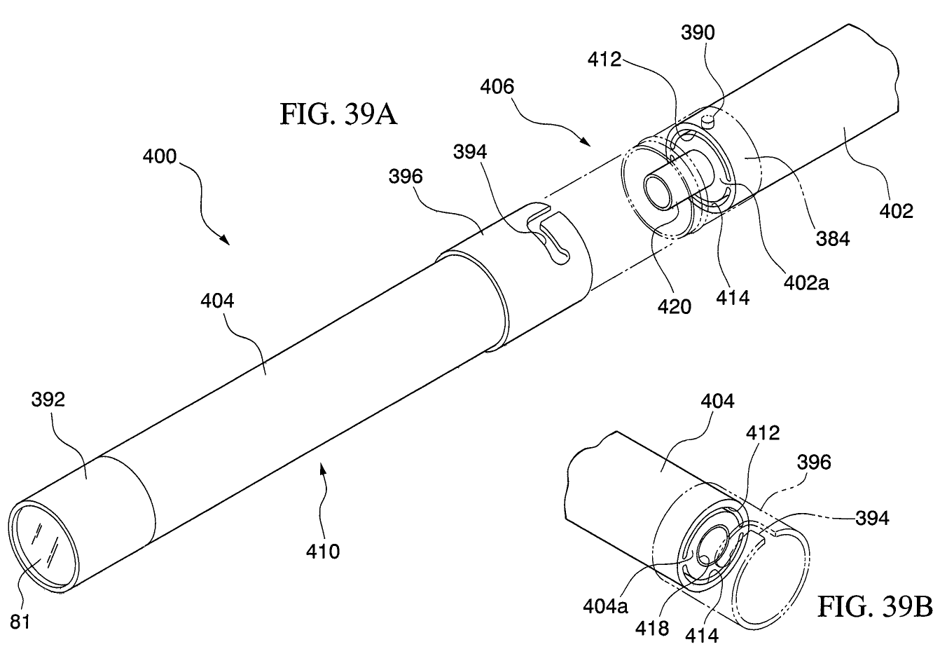

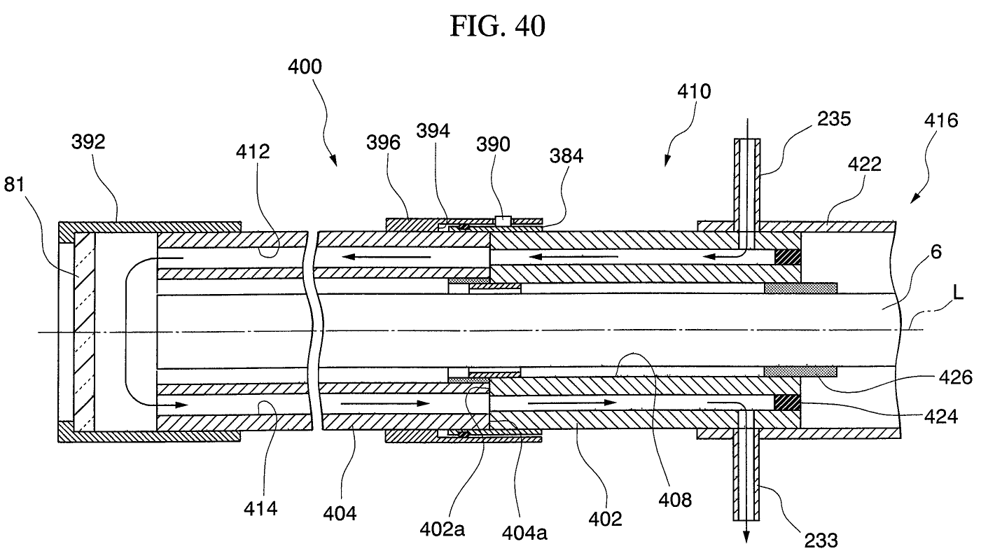

Next, a description is given for a sixteenth embodiment of the endoscope system of the present invention with reference to FIG. 39A, FIG. 39B, and FIG. 40. In the present embodiment, members common to those used in the above-described embodiment will be given the same reference numerals, the explanation of which will be omitted here.

As shown in FIG. 39A, FIG. 39B, and FIG. 40, the endoscope cooling device 400 of the present embodiment is provided with a positioning mechanism 406 for positioning the sheath main body 402 with respect to the flexible portion 404.

In place of the small flow paths 380, a supply flow path 412 for allowing a cooling fluid to flow in the direction of the distal end of the sheath 410 when connected to the flexible portion 404 and a discharge flow path 414 for allowing the cooling fluid to flow in the proximal end direction of the sheath 410 are formed around the insertion hole 408 of the sheath main body 402. The flow path 412 and the discharge flow path 414 constitute a returning flow path 416.

The positioning mechanism 406 is provided with a first short tube 418 provided at the flexible portion 404 and a second short tube 420 provided at the sheath main body 402. The first short tube 418 is provided so as to be fitted into the insertion hole 408 on the proximal end face 404a of the flexible portion 404. The second short tube 420 is provided so as to be fitted into the insertion hole 408 at the sheath main body 402 and also so as to project from to the distal end from the proximal-end face 402a of the sheath main body 402, and coupled to the inner face of the first short tube 418.

The second base 422 is provided with a fluid supply port 235 communicatively connected to the supply flow path 412 and a fluid discharge port 233 communicatively connected to the discharge flow path 414. The second base 422 is further provided with a first seal portion 424 for sealing the proximal end of the supply flow path 412 and that of the discharge flow path 414 and a second seal portion 426 for sealing the proximal end of a clearance formed between the insertion portion 6 and the insertion hole 408 upon insertion of the insertion portion 6.

Next, a description is given for actions of the endoscope and the endoscope cooling device 400 which constitute the endoscope system.

First, the main-body raised portion 390 at the third base 384 of the sheath main body 402 is engaged with the flexible portion groove 394 at the flexible portion base 396 of the flexible portion 404. At this time, the distal end face 402a of the sheath main body 402 is pressed against the proximal end face 404a of the flexible portion 404, the first short tube 418 is fitted into the second short tube 420, and the flexible portion 404 is connected to the sheath main body 402, thereby forming the sheath 410. Next, the insertion portion 6 is inserted into the insertion hole 408 and fixed in such a manner that the insertion portion 6 is separated from the inner face of the insertion hole 408. In addition, the order of assembling the sheath 410 shall not be restricted to the above description.

Next, the insertion portion 6 is inserted into a test substance, together with the sheath 410, and a cooling fluid is supplied via the fluid supply port 235 to the supply flow path 412. The cooling fluid is returned inside the distal end base 392 and discharged from the fluid discharge port 233, after flowing through the discharge flow path 414. The endoscope cooling device 400 of the present embodiment is able to provide the same effects as those of the above embodiment.

1. An endoscope cooling device for cooling an insertion portion having a bending portion that can be bent including;
a guide tube into which the insertion portion including the bending portion is inserted to form a flow path of a cooling fluid between the insertion portion and the guide tube,
wherein the guide tube is provided with a cover portion for covering the bending portion, and
other portions of the guide tube excluding the cover portion are more rigid than the cover portion.

2. The endoscope cooling device according to claim 1, wherein the cover portion is an elastic member.

3. The endoscope cooling device according to claim 1, wherein a reinforcement member for reinforcing the guide tube is provided at the other portions.

4. The endoscope cooling device according to claim 1, wherein a cooling lumen for allowing the cooling air to flow is formed at the cover portion.

5. The endoscope cooling device according to claim 1, wherein
an operating lumen to which an operating fluid is supplied is formed at the cover portion and the operating fluid is supplied to the operating lumen, thereby bending the cover portion.

6. The endoscope cooling device according to claim 1, wherein the cover portion is formed thinner than the other portions.

7. The endoscope cooling device according to claim 1, wherein a groove is formed on the outer circumferential face of the cover portion.

8. The endoscope cooling device according to claim 1, wherein
the cover portion is comprised of a tube wound into a helical shape and the cooling fluid is allowed to flow through the tube.

9. The endoscope cooling device according to claim 1, wherein
the cover portion is comprised of a tube wound in a coil shape and the cooling fluid is allowed to flow through the tube.

10. The endoscope cooling device according to claim 1, wherein
the cover portion is formed in a bellows shape.

11. The endoscope cooling device according to claim 1, which is provided with a fluid supply means for supplying the cooling fluid to the guide tube.

12. The endoscope cooling device according to claim 1, which is provided with a regulating portion for regulating the distal end portion of the insertion portion in moving with respect to the guide tube.

13. The endoscope cooling device according to claim 12, wherein
the regulating portion regulates the distal end portion in rotational movement around the central axis with respect to the guide tube.

14. The endoscope cooling device according to claim 12, wherein
the regulating portion regulates the guide tube in moving back and forth in the lengthwise direction.

15. The endoscope cooling device according to claim 12, wherein
the regulating portion is provided with a raised portion provided at one end of either the guide tube or the distal end portion and a recessed portion provided at the other end of either the guide tube or the distal end portion and engaged with the raised portion.

16. The endoscope cooling device according to claim 15, wherein
the regulating portion is provided with an auxiliary member fitted into the distal end portion and the raised portion or the recessed portion is formed in the auxiliary member.

17. The endoscope cooling device according to claim 1, wherein
the guide tube is provided with an inner sheath into which the insertion portion including the bending portion is inserted to form a first flow path of a cooling fluid between the outer circumferential face of the insertion portion and the inner circumferential face of the guide tube,
an outer sheath into which the inner sheath is inserted to form a second flow path of the cooling fluid between the outer circumferential face of the inner sheath and the inner circumferential face of the guide tube, and
the cover portion is provided respectively on the inner sheath and the outer sheath.

18. The endoscope cooling device according to claim 1, wherein
the guide tube can be divided into a plurality of portions and one or a few of the thus divided portions can be used as the guide tube.

19. An endoscope system comprising
An endoscope cooling device for cooling an insertion portion having a bending portion that can be bent; and
an endoscope having the insertion portion; wherein
the endoscope cooling device contains a guide tube into which the insertion portion including the bending portion is inserted to form a flow path of a cooling fluid between the insertion portion and the guide tube,
the guide tube contains a cover portion for covering the bending portion and
other portions of the guide tube excluding the cover portion are more rigid than the cover portion.
